SEQUENCE LISTING (4 sequences)

SEQ ID NO: 1 - protein, 15 aa, Homo sapiens
RPHFPQFSYSASGTA

SEQ ID NO: 2 - protein, 14 aa, Homo sapiens
THFPQFSYSASIRE

SEQ ID NO: 3 - protein, 16 aa, Artificial Sequence
  Synthetic Peptide
CRPHFPQFSYSASGTA

SEQ ID NO: 4 - protein, 14 aa, Artificial Sequence
  Synthetic Peptide
CTHFPQFSYSASIR

(19) United States
(12) Patent Application Publication
(10) Pub. No.: US 2017/0299606 A1
(43) Pub. Date: Oct. 19, 2017

(54) IMMUNO-MALDI TO MEASURE AKT1 AND AKT2 PHOSPHORYLATION

(71) Applicant: UVic Industry Partnerships Inc., Victoria (CA)

(72) Inventors: Robert Popp, Victoria (CA); Christoph Borchers, Victoria (CA)

(73) Assignee: UVic Industry Partnerships Inc.

(21) Appl. No.: 15/489,485

(22) Filed: Apr. 17, 2017

Related U.S. Application Data
(60) Provisional application No. 62/323,462, filed on Apr. 15, 2016.

(51) Int. Cl. G01N 33/68; G01N 33/532; G01N 33/543; G01N 33/573; G01N 33/574

(52) U.S. Cl. CPC G01N 33/6851; G01N 33/573; C12Y 304/21004; G01N 33/57415; C12Y 301/03001; G01N 33/54313; G01N 33/532; G01N 2333/91205; G01N 2560/00

(57) ABSTRACT

This application relates to methods of quantifying AKT1 and AKT2 and determining AKT1 and AKT2 phosphorylation status. The disclosed methods allow for selection of cancer therapy.

### CROSS REFERENCE TO RELATED APPLICATION

This application claims priority to U.S. Provisional Application No. 62/323,462, filed Apr. 15, 2016, which is incorporated by reference in its entirety.

### FIELD

This application relates to methods of quantifying AKT1 and AKT2 phosphorylation. The disclosed methods allow for selection of cancer therapy.

### BACKGROUND

The paradigm of personalized medicine, where patients are selected for specific treatments based on molecular signatures, has resulted in great improvements in cancer patient response. (1) However, a significant percentage of patients still only show a partial response to targeted therapies, or no response at all. In addition to tumor heterogeneity (2), this could in part be attributable to the very limited number of (mainly) genomic biomarkers used for patient stratification, which may not be accurate indicators of phenotype because of the complex regulatory mechanisms involved from DNA transcription to protein expression, as indicated by the discrepancies found between genomic, transcriptomic and proteomic data (3). Additionally, limited numbers of biomarkers cannot capture the complexity of cell signalling (4). Proteogenomics—the integration of different levels of ‘omics (genomics, transcriptomics, and proteomics) to assess molecular drivers in cancer can aid in treatment decisions, to improve patient response (5), for example to analyse the activity of cell signalling pathways.

One commonly dysregulated signaling pathway in a variety of cancers is the PI3K/AKT/mTOR pathway, which is therefore of interest as a target for therapeutic inhibition (6). Several novel inhibitors targeting this cellular pathway are currently in clinical trials, including phosphatidylinositol-3-kinase (PI3K), mechanistic target of rapamycin (mTOR), protein kinase B (AKT), and dual PI3K/mTOR inhibitors (7). The oncogene PI3K and the phosphatase and tensin homolog (PTEN) are the most commonly mutated members of this pathway (8), while AKT itself is rarely mutated in human carcinomas (9). However, quantitation of the AKT isoforms AKT1, AKT2, and AKT3 is of interest due to their overexpression and overactivation in a variety of cancers, and due to their position downstream of PI3K and PTEN (10-15). Immunohistochemistry (IHC) is commonly used to quantify signalling pathway activity and key post-translational modifications (PTMs) from tissues for patient stratification, including those of AKT (16-21). Mass spectrometry (MS)-based approaches, in particular multiple reaction monitoring-MS, have potential in quantifying proteins from clinical specimen. However, these approaches are more commonly used in clinical research.

While IHC provides histological spatial information, it suffers from the possibility of non-specificity, is difficult to multiplex, and is, at best, semi-quantitative, and the interpretation of the results is subjective. Within the past 10 years, MRM has emerged as a tool for protein biomarker analysis that can overcome the drawbacks of IHC due to its capability for multiplexed, precise, and accurate quantitative analysis of tens to hundreds of peptides per run (22-24). It is, however, costly and requires complex instrumentation, a steep learning curve and fairly long analysis times per sample, thereby limiting sample throughput, making MRM less suitable as a routine clinical tool. Whereas IHC and immunoassays are routinely used for clinical analysis of signaling pathways, multiple reaction monitoring (MRM) is more commonly used in clinical research. Both technologies have their caveats, namely the non-specificity of IHC and immunoassays, and the low throughput and complexity of MRM.

Immuno-matrix-assisted laser desorption/ionization (iMALDI) (25-27) combines the advantages of immunoassay and mass spectrometry by coupling affinity-enrichment to MALDI-time of flight (TOF) analysis. The iMALDI sample preparation is amenable to automation (28), and has analysis times of a few seconds per sample. It also has high sensitivity due to the antibody-based enrichment of proteolytic target peptides, and can be used on relatively cost-effective benchtop MALDI-TOF mass spectrometer such as the Bruker BioTyper, the Shimadzu AXIMA Microorganism Identification System, and the BioMerieux VITEK 2, which are in clinical use for microbial identification and have been cleared by the U.S. Food and Drug Administration (FDA).

### SUMMARY

To overcome the disadvantages of the current methods, a MS-based technique for the quantification of the PI3K/AKT/mTOR pathway members AKT1 and AKT2 using an iMALDI approach is disclosed herein. This high-throughput technique can be utilized for routine clinical use and for patient stratification, as well as for companion diagnostics during the drug development process.

Disclosed herein is a method of analyzing a sample obtained from a subject. The method includes: enzymatically digesting proteins in the sample, thereby producing a digested sample; dephosphorylating a portion of the digested sample, thereby producing a dephosphorylated portion of the sample and a native portion of the sample; contacting each portion of the sample with bead-antibody conjugates, wherein an antibody is specific for peptides of RAC-alpha serine/threonine-protein kinase (AKT1), RAC-beta serine/threonine-protein kinase (AKT2), or both AKT1 and AKT2, thereby producing bead-antibody conjugates bound to AKT1 and AKT2 peptides; attaching the bead-antibody conjugates bound to AKT1 and AKT2 peptides onto a solid support; washing the solid support; and detecting the AKT1 and AKT2 peptides with mass spectrometry, thereby analyzing the sample.

In an embodiment, the method further includes comparing the detected AKT1 and AKT2 peptides in the dephosphorylated portion of the sample to the AKT1 and AKT2 peptides in the native portion of the sample, thereby determining a phosphorylation status of AKT1 and AKT2.

In an embodiment, the sample is a cancer sample. For example, a colorectal cancer sample or breast cancer sample.

In an embodiment, enzymatically digesting the sample comprises contacting the sample with a proteolytic enzyme. In an example, the proteolytic enzyme is trypsin or ArgC. In an example, the ratio of the trypsin to total mass of protein in the sample in a range of 1.5:1 and 2.5:1 is used.

In an embodiments, the method further includes contacting the sample with stable-isotope-labeled standard (SIS) peptides following digesting the sample. In an example, the stable-isotope-labeled standard (SIS) peptides are isotope labeled RPHFPQFSYSASGTA (SEQ ID NO: 1) or THFPQFSYSASIRE (SEQ ID NO: 2).

In an embodiment, dephosphorylating a portion of the digested sample includes contacting the digested sample with alkaline phosphatase. In an example, the digested sample is contacted with the alkaline phosphatase for about 2 hours. In an example, the alkaline phosphatase is contacted with the digested sample at a concentration of 40-70 Units per 160 μL reaction volume.

In an embodiment, the antibody is specific for the peptides RPHFPQFSYSASGTA (SEQ ID NO: 1), or THFPQFSYSASIRE (SEQ ID NO: 2).

In an embodiment, washing the solid support includes washing the dephosphorylated portion of the sample and the native portion of the sample with ammonium citrate or ammonium phosphate. In an embodiment, the washing step is repeated three times. In an embodiments, the ammonium citrate or the ammonium phosphate is incubated with the dephosphorylated portion of the sample and the native portion of the sample at a concentration of 1-20 millimolar.

In an embodiment, the method further includes administering a therapeutically effective amount of a cancer therapeutic to the subject, if a phosphorylation status is above 10%, 15%, 20%, 25%, 30%, 35%, 40%, 45%, 50%, 55%, 60%, or more % phosphorylation for AKT1 or AKT2. In an example, the cancer therapeutic is an inhibitor of PI3K, mTOR or AKT.

In an embodiment, the sample is fresh, frozen, or formalin-fixed-paraffin embedded (FFPE). In an example, each portion of the sample contains at least 10 μg total protein.

The foregoing and other objects and features of the disclosure will become more apparent from the following detailed description, which proceeds with reference to the accompanying figures.

### BRIEF DESCRIPTION OF THE DRAWINGS

FIG. 1 is a schematic drawing showing the general iMALDI workflow. Following (1) proteolytic digestion, (2) stable isotope-labeled standard (SIS) peptides analogous to the endogenous target peptides, and (3) magnetic beads carrying target-specific anti-peptide antibodies are added to the digest solution. (4) After an enrichment period (e.g. 1 hour), the bead-antibody-peptide conjugates are washed and spotted directly onto a MALDI plate. (5) With the beads on the MALDI plate, application of acidic MALDI matrix elutes the peptides from the beads. During the drying process, the peptides co-crystallize with the MALDI matrix. (6) MALDI analysis produces mass spectra with distinct m/z ratios for endogenous (END) and SIS peptides, which allow calculation of END/SIS intensity ratios to determine the concentrations of the endogenous peptides as surrogates for the target protein.

FIG. 2A-FIG. 2F are graphs showing the quantitation of endogenous AKT1 peptide RPHFPQFSYSASGTA (SEQ ID NO: 1) from 100 μg lysate protein per replicate of (FIG. 2A) parental MDA-231 breast cancer cells, (FIG. 2B) EGF-induced MDA-231 breast cancer cells, (FIG. 2C) SW480 colon cancer cells, (FIG. 2D) HCT116 colon cancer cells, (FIG. 2E) breast tumor 70-1 and (FIG. 2F) breast tumor 70-2. The mass spectra were acquired by summing 1000 shots in positive reflector mode. 50 fmol AKT1 SIS were used as the internal standard. END=Endogenous peptide; T:P=trypsin:total protein ratio (w/w).

FIG. 3A-FIG. 3F are graphs showing AKT1 and AKT2 iMALDI assay validation results. (FIG. 3A-3C) Assessment of the linear range by spiking varying amounts of double-labelled (SIS-D) and constant SIS peptides into 10 μg E. coli digests: FIG. 3A) 1/x²-weighted linear regression. FIG. 3B) Precision of the linear regression points displayed as CVs. FIG. 3C) Error of the average linear regression points to the regression line. FIG. 3D) Accuracy results for AKT1 and AKT2 SIS-D spiked into MDA-231 breast cancer cell lysate digests. (FIG. 3E-3F) Interference testing with (FIG. 3E) parental and (FIG. 3F) EGF-induced MDA-231 breast cancer cells. END=Endogenous peptide; AKT1=empty circles; AKT2=filled circles.

FIG. 4A-FIG. 4F are mass spectra showing the captures of endogenous AKT1 peptide RPHFPQFSYSASGTA at ˜m/z 1653.9, and the corresponding, internally calibrated AKT1 SIS peptide (2 fmol/well) at m/z 1663.78 following digestion and enrichment of 10 μg lysate protein per replicate from (FIG. 4A) MDA-231 parental and (FIG. 4B) EGF-induced breast cancer cells, as well as flash-frozen tumor lysates from (FIG. 4C) HCT116 colon cancer mouse xenograft, and three different breast tumors (FIG. 4D: T-607; E: tumor 70-1; F: tumor 70-2). Digestions for spectra FIG. 4A-4D were performed at a trypsin:protein (T:P) ratio of 1:5, whereas digestions for spectra FIG. 4E and FIG. 4F were performed at a T:P ratio of 2:1. END=Endogenous peptide; SIS=stable isotope-labeled standard peptide.

FIG. 5A-FIG. 5D are mass spectra acquired for the iMALDI analysis of AKT2 from 10 μg (FIG. 5A) parental MDA-231 breast cancer cells, (FIG. 5B) EGF-induced MDA-231 breast cancer cells, (FIG. 5C) a HCT116 colon cancer mouse xenograft tumor, and (FIG. 5D) a breast tumor. END=Endogenous peptide; SIS=stable isotope-labeled standard peptide

FIG. 6 and FIG. 6B are graphs showing endogenous levels and intraday CVs determined for the endogenous (FIG. 6A) AKT1 and (FIG. 6B) AKT2, from 10 μg lysate protein of breast cancer cell lines and tumor samples, as well as an HCT116 colon cancer mouse xenograft tumor.

FIG. 7A-FIG. 7F are graphs showing the capture of synthetic AKT1 (FIG. 7A) and AKT2 (FIG. 7B) NAT and SIS peptides in PBSC buffer, and capture of digested recombinant AKT1 and AKT2 in PBSC (FIG. 7C and FIG. 7D) and E. coli lysate (FIG. 7E and FIG. 7F).

FIG. 8 is a graph showing the multiplexed analysis of digested, recombinant AKT1 and AKT2 by enriching with a 1:1 mixture of anti-AKT1 and anti-AKT2 peptide antibody-beads. END=endogenous peptide; SIS=SIS peptide.

FIG. 9A-FIG. 9F are graphs showing AKT1 and AKT2 iMALDI assay optimization. (FIG. 9A) Impact of washing MALDI spots on AKT1 SIS signal-to-noise ratio. (FIG. 9B-9D) Time-course digestion study of recombinant AKT1 and AKT2 in 100 μg E. coli lysate at 37° C.: FIG. 9B) NAT peptides quantified; * indicates a significant difference between the 1-hour digestion and other digestion periods; “n” indicates no significant difference. FIG. 9C) CVs for the replicates of the time-course digestion study; FIG. 9D) Average signal-to-noise ratios of the AKT1 and AKT2 SIS peptides. (FIG. 9E-9F) Digestion efficiency in dependence of protease inhibitor concentration and trypsin: total protein ratio during digestion tested for AKT1 in parental MDA-231 breast cancer cells. The error bars in all plots represent standard deviation. END=endogenous peptide; SIS=stable isotope-labeled standard peptide

FIG. 10 is a schematic drawing showing an exemplary overview of the workflow of the disclosed iMALDI-PPQ method. 1) Digestion of a diluted cell lysate sample. 2) Addition of SIS peptides to the digested sample. 3) The digested sample is split into two aliquots. One is incubated with alkaline phosphatase to remove any phosphate groups from peptides. 4) Antibody-beads are added to enrich the endogenous and SIS peptides of interest. 5) After a washing step, the bead-antibody-peptide complexes are spotted onto a MALDI plate. On the plate, acidic MALDI matrix is added to the dried spots which elutes the peptides from the beads. 6) MALDI analysis of both sample aliquots generates mass spectra from which the endogenous non-phosphorylated peptide levels can be calculated. The comparison of the peptide levels quantified for both sample aliquots allows calculation of the phosphorylation stoichiometry.

FIG. 11A and FIG. 11B are graphs showing titration of amount of phosphatase. FIG. 11A) Light/heavy intensity ratios of the non-phosphorylated AKT1 peptide. FIG. 11B) Light/heavy intensity ratios of the phosphorylated pS473-AKT1 peptide.

FIG. 12A and FIG. 12B are graphs showing optimization of dephosphorylation duration. Synthetic pS473-AKT1 peptide (SEQ ID NO: 1) was dephosphorylated for varying time periods (0-120 minutes) at 60 U/well at 37° C. FIG. 12A) Light/heavy intensity ratios of the non-phosphorylated AKT1 peptide. FIG. 12B) Light/heavy intensity ratios of the phosphorylated pS473-AKT1 peptide.

FIG. 13A-FIG. 13E are plots showing the quantitation of AKT1 expression levels and phosphorylation stoichiometry from an MDA-MB-231 breast cancer cell line, either (FIG. 13A) parental or (FIG. 13B) EGF-induced, and fresh frozen tissue lysates of (FIG. 13C) an HCT116 colon cancer mouse xenograft and (FIG. 13D and FIG. 13E) two breast cancer tissues; END=endogenous peptides.

FIG. 14A-FIG. 14F are graphs showing the quantitation of expression levels and phosphorylation stoichiometry for normal and adjacent tumor tissues (from FFPE samples) for two breast cancer patients. FIG. 14A and FIG. 14C) Calibration curves for AKT1 and AKT2 respectively; FIG. 14B and FIG. 14E) Endogenous peptide levels quantified for AKT1 and AKT2 respectively; FIG. 14C and FIG. 14F) Phosphorylation stoichiometry determined for quantified AKT1 and AKT2 peptides respectively. Samples used were FFPE.

FIG. 15 is a schematic drawing of AKT1 and AKT2 showing the location of the targeted peptides from AKT1 (SEQ ID NO: 1) and AKT 2 (SEQ ID NO: 2).

### SEQUENCE LISTING

The amino acid sequences listed in the accompanying sequence listing are shown using standard abbreviations for amino acids as defined in 37 C.F.R. 1.822. The sequence listing entitled 2847-96813-02 ST25 generated on Apr. 14, 2017 having a file size of 1.14 Kb is filed herewith and incorporated by reference.

SEQ ID NO: 1 is an AKT1 tryptic peptide containing a phosphorylation site.

SEQ ID NO: 2 is an AKT2 tryptic peptide containing a phosphorylation site.

SEQ ID NO: 3 is an AKT1 peptide used in antibody development.

SEQ ID NO: 4 is an AKT2 peptide used in antibody development.

### DETAILED DESCRIPTION

### List of Abbreviations

AAA, Amino acid analysis
ACN, Acetonitrile
AAA, Amino acid analysis
ACN, Acetonitrile
AKT, Protein kinase B
BCA, Bicinchoninic acid
CZE, Capillary zone electrophoresis
END, Endogenous peptide
FA, Formic acid
iMALDI, immuno-matrix assisted laser desorption/ionization
LC-MS, Liquid chromatography mass spectrometry
MRM, Multiple reaction monitoring
MS, Mass spectrometry
mTOR, Mechanistic target of rapamycin
NAT, Natural (light) version of a peptide
NMI, Natural and Medical Sciences Institute
PI3K, Phosphatidylinositol-3-kinase
PTEN, Phosphatase and tensin homolog
PTM, Post-translational modification
SIS, Stable isotope-labeled standard
TOF, Time of flight

### Terms

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2nd ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

All publications, patent applications, patents, and other references mentioned herein are incorporated by reference in their entirety for all purposes. All sequences associated with the GenBank Accession numbers mentioned herein are incorporated by reference in their entirety as were present on Apr. 17, 2017, to the extent permissible by applicable rules and/or law.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration: To provide or give a subject a therapeutic intervention, such as a therapeutic drug, procedure, or protocol (e.g., for a subject with breast, lung, or other cancer). Exemplary routes of administration for drug therapy include, but are not limited to, oral, injection (such as subcutaneous, intramuscular, intradermal, intraperitoneal, intratumoral, and intravenous), sublingual, rectal, transdermal, intranasal, and inhalation routes.

AKT1: Also known as RAC-alpha serine/threonine-protein kinase (e.g. OMIM 164730; UniProt P31749); and Protein Kinase B (PKB) alpha. AKT1 is enzyme that in humans is encoded by the AKT1 gene. This enzyme belongs to the AKT subfamily of serine/threonine kinases that contain SH2 (Src homology 2-like) domains and is involved in signal transduction, serine/threonine phosphorylation, apoptosis regulation and neurogenesis.

AKT1 sequences are publicly available. For example, GenBank® Accession Nos. NM_005163.2, NM_033230.2, NM_009652.3 disclose exemplary human, rat, and mouse AKT1 nucleotide sequences, respectively, and GenBank® Accession Nos. NP_005154.2, NP_150233.1, NP_033782.1 disclose exemplary human, rat, and mouse AKT1 protein sequences, respectively. One of ordinary skill in the art can identify additional AKT1 nucleic acid and protein sequences, including isoform and transcript variants, peptide fragments, and peptides containing phosphorylation sites.

AKT2: Also known as RAC-beta serine/threonine-protein kinase (e.g. OMIM 164731; UniProt P31751), and protein kinase B (PKB) beta. AKT2 is a putative oncogene encoding a protein belonging to the AKT subfamily of serine/threonine kinases that contain SH2-like (Src homology 2-like) domains which is a general protein kinase capable of phosphorylating several known proteins.

AKT2 sequences are publicly available. For example, GenBank® Accession Nos. NM_001626.5 and NM_001110208.2 disclose exemplary human and mouse AKT2 nucleotide sequences, respectively, and GenBank® Accession Nos. NP_001617.1, and NP_001318037.1 disclose exemplary human and mouse AKT2 protein sequences, respectively. One of ordinary skill in the art can identify additional AKT2 nucleic acid and protein sequences, including isoform and transcript variants, peptide fragments, and peptides containing phosphorylation sites.

Antibody: A polypeptide ligand comprising at least a light chain or heavy chain immunoglobulin variable region which specifically recognizes and binds an epitope of an antigen, such as a tumor-specific protein. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. In some examples, an antibody is specific for AKT1, AKT2, or both. In some examples, the antibody is a polyclonal, monoclonal, chimeric, or humanized antibody.

Antibodies include intact immunoglobulins and the variants and portions of antibodies well known in the art, such as Fab fragments, Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W. H. Freeman & Co., New York, 1997

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species, such as humans. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a murine antibody that specifically binds AKT1 or AKT2.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

A “human” antibody (also called a “fully human” antibody) is an antibody that includes human framework regions and all of the CDRs from a human immunoglobulin. In one example, the framework and the CDRs are from the same originating human heavy and/or light chain amino acid sequence. However, frameworks from one human antibody can be engineered to include CDRs from a different human antibody. All parts of a human immunoglobulin are substantially identical to corresponding parts of natural human immunoglobulin sequences.

Antigen (Ag): A compound, composition, or substance that can stimulate the production of antibodies or a T cell response in an animal, including compositions (such as one that includes an AKT1 or AKT2 protein or peptide thereof) that are injected or absorbed into an animal. Examples of antigens include, but are not limited to, peptides, lipids, polysaccharides, and nucleic acids containing antigenic determinants, such as those recognized by an immune cell. In some examples, an antigen includes a protein, peptide or immunogenic fragment thereof.

An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

The binding of an antibody to a target antigen or epitope thereof can be used to remove the target using the methods provided herein.

Attach: To chemically bond together, for example as in to bind a molecule to a solid support, for example covalently. In an example, a bead-antibody conjugate is attached to a solid support, such as a plate for MALDI mass spectrometry.

Bead-Antibody Conjugate: An antibody conjugated (e.g. attached) to a bead. The bead can be any bead to which an antibody can be directly or indirectly bound to each surface thereof. Beads can be magnetic, glass, or plastic beads. In an example, beads have a diameter of 1-5 μm, for example 2.8 μm. Commercially available beads include DYNABEADS® (ThermoFisher). Antibody conjugation can be by chemical cross-linking. A chemical tether or linker may be used in conjugation.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system. In one example, cancer cells are analyzed by the disclosed methods.

Cancer therapeutic: Treatment for subjects diagnosed with having, suspected of having, or likely to develop cancer or neoplastic disorders can include surgery, radiation, chemotherapy, immunotherapy, hormone therapy, stem cell transplant, or combinations thereof. Administered therapies can include antibodies or small molecules. Cancer therapies can target specific tumor cells, or aberrant cellular signaling pathways. A cancer therapeutic can be, for example, an inhibitor of AKT1 or AKT2, P13K, or mTOR.

Contact: Placement in direct physical association, including a solid or a liquid form. Contacting can occur in vitro or ex vivo, for example, by adding a reagent to a sample, or in vivo by administering to a subject.

Detect: To determine if a particular agent (such as one or more target molecules, such as phosphorylated AKT1 and/or AKT2) is present or absent, and in some example further includes quantification of the target. In specific examples, detection is assessed in counts, intensity, or area under a curve. In an example, detection is by mass spectrometry, such as iMALDI.

Dephosphorylate: The process by which phosphate groups are removed from a molecule (such as a protein or peptide) by phosphatase. Exemplary phosphatases include acid phosphatase and alkaline phosphatases. An alkaline phosphatase is a hydrolase enzyme which can dephosphorylate nucleotides, proteins, and alkaloids for example. To dephosphorylate proteins in a sample, the sample can be contacted with an alkaline phosphatase for a period of time to dephosphorylate proteins in the sample. In some examples, an alkaline phosphatase can be used in the disclosed methods at concentrations of about 40-70 Units/160 μL reaction volume, for example about 60 Units/160 μL reaction volume. In an example incubation with alkaline phosphatase can be about 0.5-2 hours, or about 1 hour.

Enzymatic Digestion: The process by which enzymes break down polymeric macromolecules, for example proteins and peptides. A proteolytic enzyme can digest proteins and peptides. Proteolytic enzymes lyse or cleave the amino acid polymers at specific sites. Exemplary proteolytic enzymes include trypsin, chymotrypsin, LysC, LysN, AspN, GluC and ArgC. Enzymatic digestion can be optimized for the particular enzyme used, concentration of enzyme used, enzyme incubation time and temperature.

Isotopes: Variants of a chemical element that differ in their number of neutrons. The number of protons is constant for a given element. The mass number of an isotope is its numbers of neutrons plus protons. For example, ¹²C, ¹³C, and ¹⁴C are all isotopes of carbon having 6, 7 and 8 respective neutrons. Some isotopes are radioactive and subject to decay at regular intervals. Stable Isotopes are non-radioactive isotopes. They can be used as labels as they can be distinguished by mass from more common isotopes (e.g., isotopes of greater natural abundance). Example stable isotopes which can be used to stably label a molecule include ²H, ¹³C, ¹⁵N, ¹⁸O, and ³⁴S.

Mammal: This term includes both human and non-human mammals (such as primates). Similarly, the term “subject” includes both human and veterinary subjects (such as cats, dogs, cows, and pigs) and rodents (such as mice and rats).

Mass spectrometry: A technique used to assess the mass and charge of molecules. A mass spectrometer manipulates ions with electrical and magnetic fields allowing for sorting and separation of molecules according to mass and charge. Typically, mass spectrometry can assess molecules by a mass-to-charge ratio (m/z). Since molecules are separated by mass, the presence of isotopes can be readily distinguished, as can additional features such a phosphorylation states. An exemplary type of mass spectrometry is MALDI-TOF: Matrix Assisted Laser Desorption/Ionization (MALDI) time of flight (TOF) mass spectrometry. MALDI-TOF mass spectrometry can be used in a positive-ion linear mode, negative-ion linear mode, or reflector modes.

Mechanistic Target of Rapamycin (mTOR): Also known as mammalian target of rapamycin and FK506-binding protein 12-rapamycin-associated protein 1 (FRAP1) (e.g. OMIM 601231). mTOR links with other proteins and serves as a core component of two distinct protein complexes, mTOR complex 1 and mTOR complex 2, which regulate different cellular processes.

mTOR sequences are publicly available. For example, GenBank® Accession NM_004958.3, NM_019906.1, NM_020009.2 disclose exemplary human, rat, and mouse mTOR nucleotide sequences, respectively, and GenBank® Accession Nos. NP_004949.1, NP_063971.1, and NP_064393.2 disclose exemplary human, rat, and mouse mTOR protein sequences, respectively. One of ordinary skill in the art can identify additional mTOR nucleic acid and protein sequences, including isoform and transcript variants, and peptide fragments.

Phosphatidylinositol 3-Kinase (PI3K): Also known as PIK3, and p110-alpha (e.g. OMIM 171834). PI3K is an enzyme capable of phosphorylating the 3 position hydroxyl group of the inositol ring of phosphatidylinositol (PtdIns).

P13K sequences are publicly available. For example, GenBank® Accession NM_181523.2, NM_022958.2, NM_181585.5 disclose exemplary human, rat, and mouse PI3K nucleotide sequences, respectively, and GenBank® Accession Nos. CAA87094.1, BAA24426.1, NP_001020126.1 disclose exemplary human, rat, and mouse PI3K protein sequences, respectively. One of ordinary skill in the art can identify additional PI3K and related kinase nucleic acid and protein sequences, including isoform and transcript variants, and peptide fragments.

Phosphorylation status: Refers to a percentage of phosphorylation sites that are phosphorylated, or whether a particular phosphorylation site is or is not phosphorylated. Phosphorylation status can include phosphorylation of total sites on a protein, or total percentage of phosphorylation across a sample of like proteins. In an example, phosphorylation status can be expressed as a percentage of total AKT1 and AKT2 peptides which are phosphorylated. Phosphorylation status can be calculated as the mass of phosphorylated AKT1 peptides in a native portion of a sample divided by the total mass of AKT1 peptides in the sample as indicated by the dephosphorylated portion of the sample; or as the mass of phosphorylated AKT2 peptides in the native portion of the sample divided by the total mass of AKT2 peptides in the sample as indicated by the dephosphorylated portion of the sample.

Sample: A biological specimen containing genomic DNA, RNA (e.g., mRNA), protein, or combinations thereof, obtained from a subject. Examples include, but are not limited to, peripheral blood, serum, plasma, urine, saliva, tissue biopsy, fine needle aspirate, surgical specimen, and autopsy material. A sample can be liquid or solid, such as from a liquid or solid tumor. In one example, a sample is a tissue sample (such as a tissue sample, such as a core biopsy) or needle biopsy (such as a fine need aspirate) from a subject suspected of having, or at risk of cancer. Samples can further be fresh, frozen, or formalin-fixed paraffin-embedded (FFPE). A sample can contain at least 10 μg total protein, at least 15 μg total protein, at least 20 μg total protein, at least 25 μg total protein, at least 30 μg total protein, at least 40 μg total protein, at least 50 μg total protein, at least 75 μg total protein, at least 100 μg total protein, or more. In some examples, a sample includes less than 100 μg total protein, less than 50 μg total protein, less than 25 μg total protein or less than 10 μg total protein, such as 100 to 10 μg total protein, 50 to 10 μg total protein, or 20 to 10 μg total protein.

In some examples, samples are used directly in the methods provided herein. In some examples, samples are manipulated prior to analysis using the disclosed methods, such as through concentrating, filtering, centrifuging, diluting, desalting, denaturing, reducing, alkylating, proteolyzing, or combinations thereof. In some examples, components of the samples are isolated or purified prior to analysis using the disclosed methods, such as isolating cells, proteins, and/or nucleic acid molecules from the samples.

Specifically binds: refers to the ability of individual antibodies to specifically immunoreact with an antigen, such as peptide or protein. In an example, an antibody specifically binds to an AKT1 or AKT2 peptide, or both peptides.

The binding is a non-random binding reaction between an antibody molecule and an antigenic determinant of the T cell surface molecule. The desired binding specificity is typically determined from the reference point of the ability of the antibody to differentially bind the T cell surface molecule and an unrelated antigen, and therefore distinguish between two different antigens, particularly where the two antigens have unique epitopes. An antibody that specifically binds to a particular epitope is referred to as a “specific antibody”.

In some examples, an antibody specifically binds to a target (such as an AKT1 or AKT2 peptide, or both) with a binding constant that is at least 10³ M⁻¹ greater, 10⁴ M⁻¹ greater or 10⁵ M⁻¹ greater than a binding constant for other molecules in a sample or subject. In some examples, an antibody (e.g., monoclonal antibody) or fragments thereof, has an equilibrium constant (Kd) of 1 nM or less. For example, an antibody binds to a target, such as tumor-specific protein with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸ M, or at least about 2.0×10⁻⁸ M. Kd values can, for example, be determined by competitive ELISA (enzyme-linked immunosorbent assay) or using a surface-plasmon resonance device such as the Biacore T100, which is available from Biacore, Inc., Piscataway, N.J.

Solid Support: The solid support which forms a matrix for assay spotting can be formed from known materials, such as any water immiscible material. In some examples, suitable characteristics of the material that can be used to form the solid support surface include: being amenable to surface activation such that upon activation, the surface of the support is capable of covalently attaching an antibody that can bind to the target agent (such as AKT1, AKT2, or both) with high specificity; being chemically inert such that at the areas on the support not occupied by the molecule can bind to the agent with high specificity are not amenable to non-specific binding, or when non-specific binding occurs, such materials can be readily removed from the surface without removing antibody.

The surface of a solid support may be activated by chemical processes that cause covalent linkage of an agent (e.g., antibody specific for AKT1, AKT2, or both) to the support. However, any other suitable method may be used for immobilizing an agent (e.g., antibody) to a solid support including, without limitation, ionic interactions, hydrophobic interactions, covalent interactions and the like. The particular forces that result in immobilization of a recognition molecule on a solid phase are not important for the methods and devices described herein.

In one example the solid support is a particle, such as a bead. Such particles can be composed of metal (e.g., gold, silver, platinum), metal compound particles (e.g., zinc oxide, zinc sulfide, copper sulfide, cadmium sulfide), non-metal compound (e.g., silica or a polymer), as well as magnetic particles (e.g., iron oxide, manganese oxide). In some examples the bead is a latex or glass bead. The size of the bead is not critical; exemplary sizes include 5 nm to 5000 nm in diameter. In one example such particles are about 1 μm in diameter.

In another example, the solid support is a bulk material, such as a paper, membrane, porous material, water immiscible gel, water immiscible ionic liquid, water immiscible polymer (such as an organic polymer), and the like. For example, the solid support can comprises a membrane, such as a semi-porous membrane that allows some materials to pass while others are trapped. In one example the membrane comprises nitrocellulose

In one example, the solid support is composed of an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof). In one example, a solid support is composed of glass, or glass coated with Indium Tin Oxide (ITO). In one example, a solid support is composed of stainless steel.

In yet other examples, the solid support is a material containing, such as a coating containing, any one or more of or a mixture of the ingredients provided herein.

A wide variety of solid supports can be employed in accordance with the present disclosure. Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

In one example solid support is a plate for use in MALDI mass spectrometry. A MALDI plate may be a commercially available 96- or 384-spot plate, for example μFocus MALDI plates by Hudson Surface Technology (New Jersey, USA). MALDI plates may be subjected to sample spotting, drying, incubation with MALDI matrix, washing etc.

Stable Isotope Labelled Standard: A molecule used as an assay standard that includes or contains one or more stable isotopes (such as 1, 2, 3, 4 or 5 stable isotopes). A labelled molecule, such as a labeled target molecule (such as AKT1, AKT2, or both), may be distinguished from its unlabeled form by a difference in mass, e.g., by mass spectrometry. Stable isotope labeled molecules can be used as stable isotope labeled standard (SIS) molecules, for purposes of assay calibration. Example stable isotopes used for labelling are ²H, ¹³C, ¹⁵N, ¹⁸O, and ³⁴S. The terms “stable isotope labeled standard” and “SIS” are used interchangeably herein. Stable Isotope Labeled Standards can include, for example, stable-isotope labeled arginine, or phenylalanine, or both.

An unlabeled form of a standard isotope labelled molecule may have the same chemical structure as its stable isotope labeled counterpart but be comprised of unmodified elements with standard isotope numbers. For example, an unlabeled molecule can include standard elements (e.g., ¹H, ¹²C, ¹⁴N, ¹⁶O, or ³²S) whereas the stable isotope labeled molecule can include one or more isotopes (e.g., ²H, ¹³C, ¹⁵N, ¹⁸O, and ³⁴S). Thus, a molecule in its unlabeled (e.g., native) form will have a distinguishable mass from its standard isotope labeled version.

Subject: Includes both human and veterinary subjects, such as humans, non-human primates, pigs, sheep, cows, rodents, birds, and the like, which can be the source of a test sample analyzed by the disclosed methods. An “animal” is a living, multi-cellular vertebrate organisms, a category that includes, for example, mammals and birds (e.g., chickens). The term mammal includes both human and non-human mammals. In two non-limiting examples, a subject is a human subject or a murine subject. In some examples, the subject has, or is suspected of having, cancer.

Therapeutically effective amount: An amount of a pharmaceutical preparation that alone, or together with a pharmaceutically acceptable carrier or one or more additional therapeutic agents, induces the desired response. A therapeutic agent, such as an anti-neoplastic chemotherapeutic agent, radiotherapeutic agent, or biologic agent, is administered in therapeutically effective amounts.

Therapeutic agents can be administered in a single dose, or in several doses, for example daily, during a course of treatment. However, the effective amount can be dependent on the source applied, the subject being treated, the severity and type of the condition being treated, and the manner of administration. Effective amounts of a therapeutic agent can be determined in many different ways, such as assaying for a sign or a symptom of a cancer. Effective amounts also can be determined through various in vitro, in vivo or in situ assays. For example, a pharmaceutical preparation can decrease one or more symptoms of a cancer, for example, a decrease in the size of the cancer, the number of tumors, the number of metastases, or other symptoms (or combinations thereof) by at least 20%, at least 50%, at least 70%, at least 90%, at least 98%, or even 100%, as compared to an amount in the absence of the pharmaceutical preparation.

Treating a disease: “Treatment” refers to a therapeutic intervention that ameliorates a sign or symptom of a disease or pathological condition, such a sign or symptom of cancer. Treatment can also induce remission or cure of a condition, or can reduce the pathological condition, such as a reduction in tumor size and/or volume, a reduction in tumor burden, a reduction in a sign or a symptom of a tumor (such as cachexia), a reduction in metastasis, or combinations thereof. In particular examples, treatment includes preventing a disease, for example by inhibiting the full development of a disease, such as decreasing the ability of a tumor to metastasize. Prevention of a disease does not require a total absence of disease.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Exemplary tumors, such as cancers, that can be analyzed and treated with the disclosed methods include solid tumors, such as breast carcinomas (e.g. lobular and duct carcinomas), sarcomas, carcinomas of the lung (e.g., non-small cell carcinoma, large cell carcinoma, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, and lymphatic tumors (including B-cell and T-cell malignant lymphoma). In one example, the tumor is an adenocarcinoma.

The methods can also be used in analysis and treatment of liquid tumors, such as a lymphatic, white blood cell, or other type of leukemia. In a specific example, the tumor analyzed is a tumor of the blood, such as a leukemia (for example acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, and adult T-cell leukemia), lymphomas (such as Hodgkin's lymphoma and non-Hodgkin's lymphoma), and myelomas).

### Overview

To overcome the drawbacks of prior clinical diagnostic approaches, immuno-Matrix Assisted Laser Desorption/Ionization (iMALDI) assays with automated liquid handling for the quantitation of AKT1 and AKT2 is described herein. This disclosed methods are suitable for detecting AKT1 and AKT2 by iMALDI. The methods provide highly sensitive and reproducible quantitation of AKT1 and AKT2 and adaptable to use with common clinical equipment, for example a benchtop MALDI mass spectrometer.

Provided herein are iMALDI assays for the accurate, sensitive and precise quantitation of the PI3K/AKT/mTOR pathway members, AKT1 and AKT2, from a total of only ˜25 μg total lysate protein (10 μg per analysis). This assay format overcomes the non-specificity of IHC and immunoassays, and in comparison with targeted LC-MS approaches, utilizes simpler instrumentation that is already in clinical laboratories, has increased sample throughput, and allows analysis of samples that are available only at very low quantities, such as core needle biopsies.

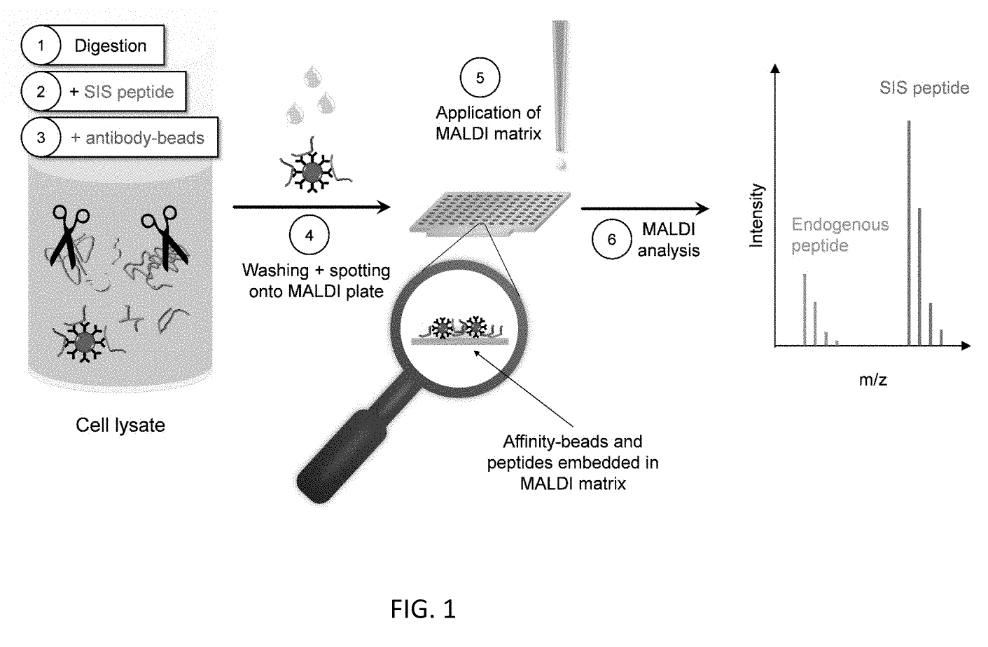

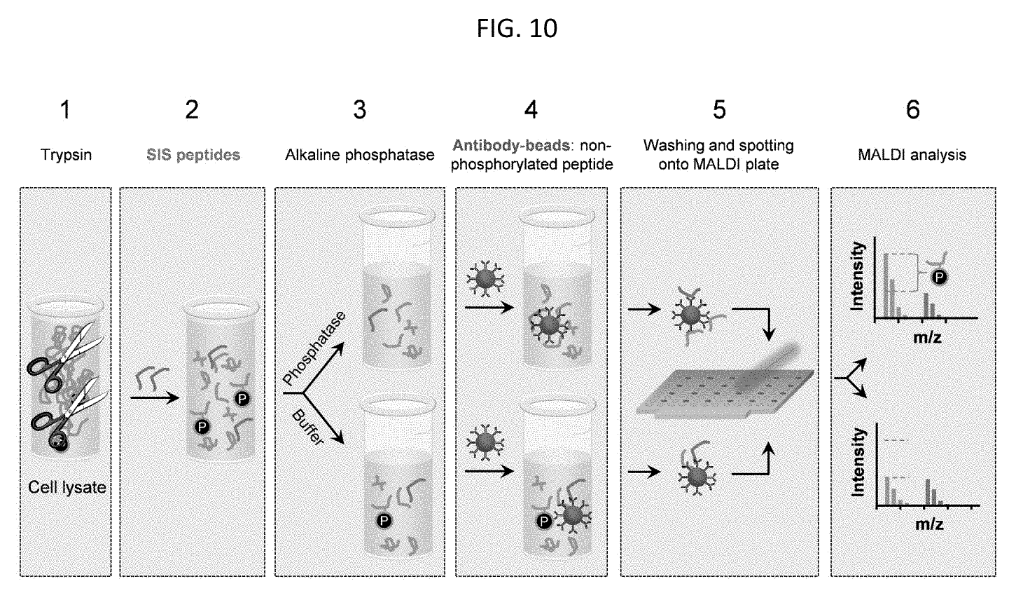

The method quantifies AKT1 and AKT2 based on their C-terminal tryptic peptides, which encompass phosphorylation sites that are crucial for complete kinase activation. The disclosed iMALDI method for quantifying proteins involved in cell signaling from miniature sample amounts can be used in companion diagnostics during drug development, as well for patient selection, to monitor treatment response, and to provide inside into mechanisms related to drug resistance. An iMALDI workflow is outlined in FIG. 1. An embodiment of the disclosed methods further quantifies phosphorylation status of target peptides. A workflow for this embodiment is outlined in FIG. 10. The steps of each are described herein.

The disclosed methods provide for quantifying AKT1 and AKT2 in a subject sample. Furthermore, the disclosed methods allow for determining a phosphorylation status of AKT1 and AKT2. The disclose methods employ utilized iMALDI, a method for selectively enhancing a sample for target peptides (e.g. AKT1 and AKT2) with a selective antibody prior to quantification with mass spectrometry (e.g. MALDI mass spectrometry).

The disclose methods can be applied to a tissue sample or a subject suspected of or diagnosed with having a cancer. AKT1 and AKT2 are involved in the PI3K/mTOR/AKT pathway that is dysregulated in a variety of wide variety of cancers. The disclosed methods allow for quantification of AKT1 and AKT2 and also of quantification of phosphorylation of both proteins.

The methods can be applied to a sample from a subject. The sample can be a tissue sample, for example a liquid or solid tumor sample. In an example, the sample is harvested by needle biopsy. The sample can further be fresh, frozen of FFPE treated. Methods are provided herein to extract protein from FFPE samples such that following protein extraction samples are quantified using the sample protocol.

In an example, samples are subjected to enzymatic digestion. Enzymatic digestion uses proteolytic enzymes to cleave proteins or peptides into fragments at predictable cleavage sites. The present methods use peptide fragments of the larger AKT1 and AKT2 proteins as proxies for total protein concentration as the smaller fragments are more readily quantified by mass spectrometry. Example proteolytic enzymes include trypsin and ArgC.

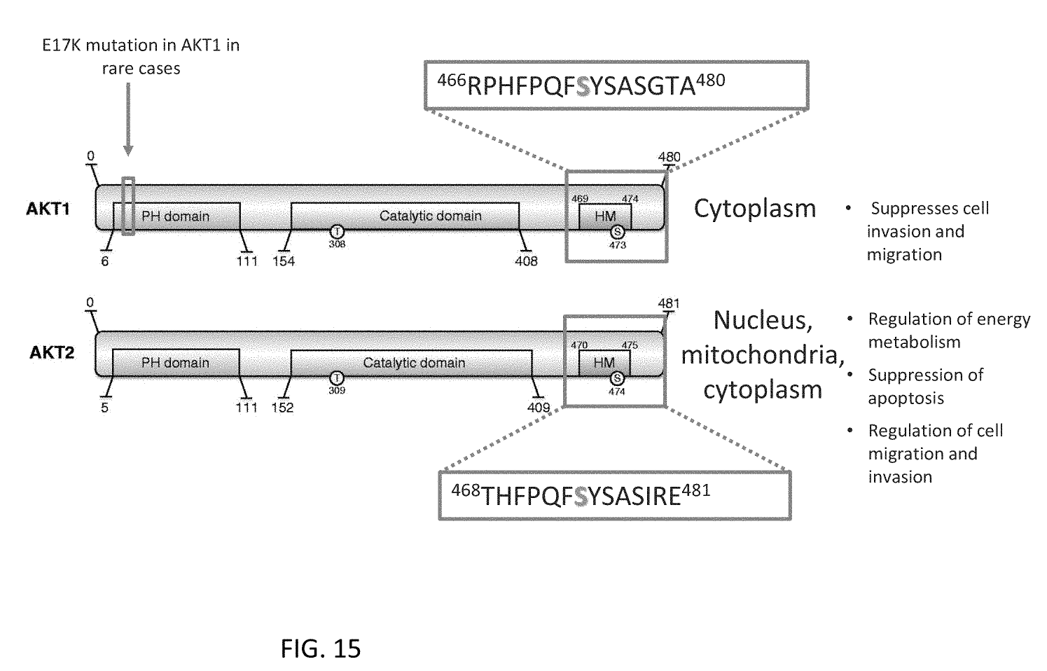

Specific peptide fragments can be targeted for quantification. In an example, a peptide is targeted for its size and for its amino acid contents. In an example, peptides produced by trypsin or ArgC digestion are of a suitable size for quantification by MALDI mass spectrometry. In a further example, a peptide targeted for mass spectrometry can contain amino acid sites of interest, for example conserved sites presented in a variety of isoforms that may be present in a sample. In a further example, targeted peptides may contain amino acids of interest, for example phosphorylation sites. Example target peptides for AKT1 and AKT2 are shown in FIG. 15. In an example, an AKT1 tryptic peptide containing a phosphorylation site is RPHFPQFSYSASGTA (SEQ ID NO: 1). In an example, an AKT2 tryptic peptide containing a phosphorylation site is THFPQFSYSASIRE (SEQ ID NO: 2).

The methods disclosed include the addition of stable isotope labeled standard (SIS) peptides. These peptides are distinguishable by mass from native peptides containing only isotopes in their natural abundances. The SIS peptides can include, for example, stable-isotope labeled arginine, or phenylalanine, or both. SIS standards can be include the peptides of SEQ ID NO: 1 and SEQ ID NO: 2 with the incorporation of stable isotope-coded arginine and phenylalanine residues (for example including ¹³C, ¹⁵N, or both). In an alternate example, whole proteins can be stable isotope labelled and added to the sample prior to enzymatic digestion.

Following addition of SIS standards, the digested sample may be divided to assess a phosphorylation status of AKT1 and AKT2, see Step 2) of FIG. 10. One portion of the sample is subjected to dephosphorylation prior to quantification with mass spectrometry. The remaining portion of the sample is left in its native state. Comparing the dephosphorylated portion of the sample with the native portion of the sample allows for calculation of a percentage phosphorylation of each peptide.

Both native and dephosphorylated portions of the sample are contacted with bead-antibody conjugates, wherein an antibody is specific for peptides of AKT1, AKT2, or both AKT1 and AKT2, thereby producing bead-antibody conjugates bound to AKT1 and AKT2 peptides. In some examples, the antibodies are monoclonal or polyclonal, or a fragment thereof. In an example, the antibodies specifically bind to SEQ ID NO: 1 and SEQ ID NO: 2 respectively, or can be cross-reactive for both peptides. The antibodies can be conjugated to magnetic beads (e.g. DYNABEADS®). The bead-antibody conjugates can bind the target peptides such that the sample may be enriched for those peptides, for example by washing away unbound peptides.

In an embodiment, the enriched sample is spotted onto a solid support. An example solid support is a plate for use in MALDI mass spectrometry. A MALDI plate may be a commercially available 96- or 384-spot plate, for example μFocus MALDI plates by Hudson Surface Technology (New Jersey, USA). MALDI plates may be subjected to sample spotting, drying, incubation with MALDI matrix, washing etc. In an example, samples (such as the bead-antibody-peptide complexes) are spotted onto a MALDI plate then allowed to dry. An acidic MALDI matrix is added on top of the spotted MALDI plate. In an embodiments, the acidic pH disrupts the antibody-peptide bonds. As the matrix dries down, the loose peptides co-crystallize with the matrix molecules. Thus, in an embodiment, antibody-bead conjugates are not covalently attached to the plate, but instead dry and stay on the plate. In an example, the MALDI plate is then washed to remove any compounds (e.g., salts), such as that lower the ionization efficiency of the target compounds prior to mass spectrometry. Such a wash step can use ammonium citrate or ammonium phosphate. In an example, a concentration of ammonium citrate or ammonium phosphate is about 0-40 millimolar, about 1-20 millimolar, about 1-15 millimolar, about 1-10 millimolar, about 5-10 millimolar, about 6 millimolar, about 7 millimolar, about 8 millimolar, about 9 millimolar, or about 10 millimolar. In an example, a washing step may include multiple washes of about 2-10 seconds each, for example about 2, 3, 4, or 5 washes. In an example, washing a MALDI plate occurs at room temperature.

The sample spotted onto a solid support can then be analyzed by mass spectrometry. MALDI analysis can distinguish between SIS labelled peptides and native peptides. Furthermore, the difference in mass between phosphorylated and dephosphorylated peptides can further be distinguished. MALDI mass spectrometry can be used in positive-ion linear mode or reflector mode.

A phosphorylation status can be determined by MALDI analysis comparing the native and dephosphorylated portions of the sample. In an example, phosphorylation status can be expressed as a percentage of total AKT1 and AKT2 peptides which are phosphorylated. Phosphorylation status can be calculated as the mass of phosphorylated AKT1 peptides in a native portion of a sample divided by the total mass of AKT1 peptides in the sample as indicated by the dephosphorylated portion of the sample; or as the mass of phosphorylated AKT2 peptides in the native portion of the sample divided by the total mass of AKT2 peptides in the sample as indicated by the dephosphorylated portion of the sample.

In an embodiment, phosphorylation status can be used in determining a prescribed therapy. In an embodiment, elevated phosphorylation status of AKT1, AKT2, or both can be indicative of aberrant PI3K/mTOR/AKT signaling. In an embodiment, a patient exhibiting phosphorylation levels of AKT1, AKT2, or both above 10%, 15%, 20%, 25%, 30%, 35%, 40%, 45%, 50%, 55%, 60%, or more are candidates for therapy with aPI3K/AKT/mTOR pathway inhibitor.

### Samples

A sample used for analysis can be a sample from a suspect diagnosed with, or suspected of having cancer (e.g., breast cancer or colorectal cancer). A sample can be liquid or solid, such as from a liquid or solid tumor. In one example, a sample is a tissue sample, core sample, or needle biopsy from a subject suspected of having, or at risk of cancer. Samples can further be fresh, frozen, or formalin-fixed paraffin-embedded (FFPE). A sample can contain at least 10 μg total protein, at least 15 μg total protein, at least 20 μg total protein, at least 25 μg total protein, at least 30 μg total protein, at least 40 μg total protein, at least 50 μg total protein, at least 75 μg total protein, at least 100 μg total protein, or more, such as 10 to 1000 μg total protein, 10 to 500 μg total protein, 10 to 100 μg total protein, 10 to 50 μg total protein or 10 to 25 μg total protein.

The disclosed methods can vary in the range of detection for AKT1 and AKT2. In embodiments, the range of detection for AKT1 per total sample volume is about 1-300 pg/μg, about 1-200 pg/μg, about 1-150 pg/μg, about 1-125 pg/μg, about 2.0-120 pg/μg, or about 2.8-111 pg/μg. In embodiments, the range of detection for AKT2 per total sample volume is about 1-300 pg/μg, about 1-200 pg/μg, about 1-150 pg/μg, about 1-125 pg/μg, about 2.0-120 g/μg, or about 2.6-102 pg/μg.

The disclosed methods have a lower limit of detection (LLOQ) of at least 0.2 fmol of protein, 0.4 fmol of protein, 0.5 fmol of protein, 0.6 fmol of protein, 0.7 fmol of protein, 0.8 fmol of protein, 0.9 fmol of protein, or more. The disclosed methods have an upper LOQ (ULOQ) of about 15 fmol of peptide, 16 fmol of peptide, 17 fmol of peptide, 18 fmol of peptide, 19 fmol of peptide, or 20 fmol of peptide, or more on the solid support.

In some examples, samples are used directly. In some examples, samples are manipulated prior to analysis using the disclosed methods, such as through concentrating, filtering, centrifuging, diluting, desalting, denaturing, reducing, alkylating, proteolyzing, or combinations thereof. In some examples, components of the samples are isolated or purified prior to analysis using the disclosed methods, such as isolating proteins from the samples.

In an example where a sample is FFPE treated additional steps can be taken prior to enzymatic digestion. FFPE samples can be treated to extract proteins contained in the sample and to remove residual paraffin and formalin. For examples, samples can be first deparaffinized. Larger FFPE samples, for example, FFPE tissue microarray (TMA) cores can be subjected to freezing and homogenizing prior to deparaffinization. For example, and FFPE TMA core can be frozen with liquid nitrogen and ground with a pestle to produce a powder that can be resuspended.

Following deparaffinization of FFPE samples and rehydration, proteins can be extracted. An example protocol applied to 1 mL volume of rehydrated, deparaffinized protein includes the addition of 150 μL of the protein extraction buffer (0.05 M TrisHCl, pH 8.1, 2% sodium deoxycholate, 10 mM TCEP, 1× Halt protease and phosphatase inhibitor cocktail) was added to the rehydrated tissue sample. The tube is incubated on ice for 5 min, followed by brief vortexing. On a Thermomixer, the tube is then incubated at 900 rpm at 99° C. for 20 min, then at 80° C. for 2 hours. After the incubation, the tube is placed on ice for 1 min. Next, the tube is centrifuged for 15 min at 14,000×g at 4° C. The supernatant is transferred to a new 1.5 mL microfuge tube. A 25 μL aliquot is stored in a separate tube at −80° C. until total protein content is determined by reducing agent-compatible bicinchoninic acid BCA assay (Thermo Fisher).

### Enzymatic Digestion

Proteolytic enzymes are used to digest the test sample into protein fragments of suitable size for MALDI analysis. Enzymatic digestion can be performed to achieve complete digestion without excess proteolytic enzyme. Excessive proteolytic enzyme in the sample can result in enzyme self-digestion and the creation of excess peptides which can interfere with MALDI analysis. In an embodiment, excess proteolytic enzyme in the sample can result in non-specific MALDI peaks. Exemplary proteolytic enzymes include trypsin, chymotrypsin, LysC, LysN, AspN, GluC and ArgC.

In an embodiment, trypsin is used for enzymatic digestion. Trypsin produces peptides of a size suitable for MALDI analysis. In an example, and AKT1 tryptic peptide containing a phosphorylation site is RPHFPQFSYSASGTA (SEQ ID NO: 1). In an example, an AKT2 tryptic peptide containing a phosphorylation site is THFPQFSYSASIRE (SEQ ID NO: 2).

In an embodiment, a ratio of the trypsin to total mass of protein in the sample is used at a range of 1.5:1 to 2.5:1, for example about 1.5:1, about 2:1, or about 2.5:1. In an embodiment, the sample is incubated with trypsin for an enzymatic digestion of about at least 30 min, at least 60 min, at least 90 min, or at least 120 min, such as 0.5-2 hours, or about 0.5 hours, about 45 minutes, about 1 hour, about 1.25 hour, about 1.5 hours, about 1.75 hour, or about 2 hours. In an embodiment, incubation with trypsin occurs at about 37° C.

### Stable Isotope Labeled Standards

A Stabled Isotope Labelled Standard (SIS) molecule can be used as an assay standard for the disclosed methods. SIS molecules can include or contain one or more stable isotopes (such as 1, 2, 3, 4 or 5 stable isotopes). Stable isotope labeled molecules can be used as stable isotope labeled standard (SIS) molecules, for purposes of assay calibration. An SIS peptide can be distinguished from the same peptide containing only isotopes of natural abundance, e.g., by mass spectrometry. Example stable isotopes used for labelling are ²H, ¹⁵N, ¹⁸O, and ³⁴S.

The methods disclosed include the addition of stable isotope labeled standard (SIS) peptides. These peptides are distinguishable by mass from native peptides containing only isotopes in their natural abundances. The SIS peptides can include, for example, stable-isotope labeled arginine, or phenylalanine, or both. SIS standards can include the peptides of SEQ ID NO: 1 and SEQ ID NO: 2 with the incorporation of stable isotope-coded arginine and phenylalanine residues (for example including ¹³C, ¹⁵N, or both). In an alternate example, whole proteins can be stable isotope labelled and added to the sample prior to enzymatic digestion.

### Dephosphorylation

To assess a phosphorylation status of AKT1 and AKT2, the sample is divided into separate portions prior to further analysis. Of the divided portions, one portion can be subjected to dephosphorylating and one portion can be kept in its native phosphorylation state. Dephosphorylation is the process by which phosphate groups are removed from a molecule by phosphatase. In some examples, one portion of the sample is treated to achieve complete dephosphorylation.

Exemplary phosphatases can include acid phosphatase and alkaline phosphatase. An alkaline phosphatase can dephosphorylate nucleotides, proteins, and alkaloids for example.

To dephosphorylate a portion of the sample, the portion of the sample can be contacted with an alkaline phosphatase under conditions sufficient to dephosphorylate AKT1 and AKT2 peptides or proteins in the sample. In some examples, an alkaline phosphatase is used at concentrations of at least 30 U/160 μL sample volume, at least 40 U/160 μL sample volume, or at least 60 U/160 μL sample volume, such as about 40-70 Units/160 μL sample volume, for example about 60 Units/160 μL sample volume, or 0.375 Units/μL. In an embodiment, the concentration of phosphatase is 40-70 U/10 μg total protein, for example 60 U/10 μg total protein. In an example, 20 μg of alkaline phosphatase in 160 μL sample volume provides a concentration of about 60 Units/160 μL. This concentration can vary with the source, lot, age, storage conditions, and activity of the phosphatase.

In an example an incubation with alkaline phosphatase can be at least 30 min, at least 60 min, or at least 120 min, such as about 0.5-2 hours, or about 30 min, about 45 minutes, about 1 hour, about 1.25 hour, about 1.5 hours, about 1.75 hour, or about 2 hours. In an example, dephosphorylation occurs at room temperature.

### Affinity Enrichment

Samples used in the disclosed methods can be enriched for target molecules (e.g. AKT1 and AKT2 peptides). Enriching for these peptides can be achieved by selection with affinity molecules, for example antibodies. Antibodies can specifically bind to AKT1 or AKT2 peptides, or both (e.g., a bi-specific antibody that can specifically bind AKT1 and AKT2). In some examples, an antibody specifically binds to a target (such as an AKT1 or AKT2 peptide) with a binding constant that is at least 10³ M⁻¹ greater, 10⁴ M⁻¹ greater or 10⁵ M⁻¹ greater than a binding constant for other molecules in a sample or subject. In some examples, an antibody (e.g., monoclonal antibody) or fragments thereof, has an equilibrium constant (Kd) of 1 nM or less. For example, an antibody binds to a target, such as tumor-specific protein with a binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸ M, or at least about 2.0×10⁻⁸ M. Kd values can, for example, be determined by competitive ELISA (enzyme-linked immunosorbent assay) or using a surface-plasmon resonance device such as the Biacore T100, which is available from Biacore, Inc., Piscataway, N.J.

Antibodies can specifically bind to RPHFPQFSYSASGTA (SEQ ID NO: 1) and AKT1 peptide, or THFPQFSYSASIRE (SEQ ID NO: 2), an AKT2 peptide. Antibodies that specifically bind to these peptides can further specifically bind to CRPHFPQFSYSASGTA (SEQ ID NO: 3) and CTHFPQFSYSASIR (SEQ ID NO: 4) which contain an n-terminal cysteine used to link the peptide to a carrier protein for antibody development. Antibodies used to enrich samples in the disclosed methods may further be cross-reactive for peptides of both AKT1 and AKT2.

Antibodies used in affinity enrichment for the disclosed methods can be tethered to a bead, for example as a bead-antibody conjugate. The bead can be any bead to which an antibody can be directly or indirectly bound to each surface thereof. Beads can be magnetic beads. In an example, beads have a diameter of 1-5 μm, for example 2.8 μm. Commercially available beads include DYNABEADS® (ThermoFisher). Antibody conjugation can be by chemical cross-linking. A chemical tether or linker may be used in conjugation.

### Solid Support

In an embodiment, the enriched samples is spotted onto a solid support. In one example, a solid support is composed of stainless steel. An example solid support is a plate for use in MALDI mass spectrometry. A MALDI plate may be a commercially available 96- or 384-spot plate, for example μFocus MALDI plates by Hudson Surface Technology (New Jersey, USA). MALDI plates may be subjected to sample spotting, drying, incubation with MALDI matrix, washing etc. In an example, samples are spotted onto a MALDI plate then allowed to dry. The spotted MALDI plate is then incubated with MALDI matrix, which in some examples allows for elution of antibody-bead conjugates onto the solid support of the MALDI plate.

In an example, samples are spotted onto a MALDI plate then allowed to dry. The spotted MALDI plate is then incubated with MALDI matrix, which can allow for elution of antibody-bead conjugates onto the solid support of the MALDI plate. The acidic MALDI matrix is added on top. In an embodiments, the acidic pH disrupts the antibody-peptide bonds. As the matrix dries down, the loose peptides co-crystallize with the matrix molecules. Thus, in an embodiment, antibody-bead conjugates are not covalently attached to the plate. In an example, the MALDI plate is then washed to remove any compounds (e.g., salts) that would lower the ionization efficiency of the target compounds prior during mass spectrometric analysis. Such a wash step can use ammonium citrate or ammonium phosphate. In an example, a concentration of ammonium citrate or ammonium phosphate is about 0-40 millimolar, about 1-20 millimolar, about 1-15 millimolar, about 1-10 millimolar, about 5-10 millimolar, about 6 millimolar, about 7 millimolar, about 8 millimolar, about 9 millimolar, or about 10 millimolar. In an example, a washing step may include multiple washes of about 2-10 seconds each, for example about 2, 3, 4, or 5 washes. In an example, washing a MALDI plate occurs at room temperature. In an example, washing with water or an acidic wash is not sufficient.

### Detecting AKT1 and AKT2

Detecting the AKT1 and AKT2 peptides from the sample can be done with mass spectrometry, for example MALDI mass spectrometry. A mass spectrometer manipulates ions with electrical and magnetic fields allowing for sorting and separation of molecules according to mass and charge. Typically, mass spectrometry can assess molecules by a mass-to-charge ratio (m/z). Since molecules are separated by mass, the presence of isotopes can be readily distinguished, as can additional features such a phosphorylation states. An example type of mass spectrometry is MALDI-TOF: Matrix Assisted Laser Desorption/Ionization (MALDI) time of flight (TOF) mass spectrometry. MALDI-TOF mass spectrometry can be used in a positive-ion linear mode or a positive ion reflector mode. In an example, positive-ion mode can be suited to the charge state of the AKT1 and AKT2 ions generated.

### Assay Accuracy

The accuracy of the methods disclosed herein can be quantified by comparison to added SIS peptides. In an example, analytical accuracy can be determined by using comparison with known concentrations of SIS peptides spiked into control samples. Accuracy can then be calculated as a percentage of SIS peptide quantified by the disclosed methods compared to the total amount of SIS peptide spiked into the control. Accuracy can be assessed using these methods for SIS AKT1 peptides and SIS AKT2 peptides.

In embodiments, the accuracy of a disclosed method for quantification of AKT1 is at least 80%, 81%, 82%, 83%, 84%, 85%, 86%, 87%, or more. In embodiments, the accuracy of the method for quantification of AKT1 is in the range of 80-95%, about 85-90%, or about 87-89%. In embodiments, the accuracy of a disclosed method for quantification of AKT2 is at least 85%, 86%, 87%, 88%, 89%, 90%, 91%, 92%, 93%, 94%, 95%, 96%, 97%, 98%, or more. In embodiments, the accuracy of a disclosed method for quantification of AKT2 is in the range of 85-99%, about 90-99%%, or about 92-98%.

### Determination of Phosphorylation Status

The methods provided herein can detect or measure the phosphorylation status of AKT1 and AKT2 in a test sample, such as a cancer sample. Phosphorylation status can be expressed as a percentage of total AKT1 and AKT2 peptides are phosphorylated. In an example, phosphorylation status can be expressed as a percentage of total AKT1 and AKT2 peptides which are phosphorylated. Phosphorylation status can be calculated as the mass of phosphorylated AKT1 peptides in a native portion of a sample divided by the total mass of AKT1 peptides in the sample as indicated by the dephosphorylated portion of the sample; or as the mass of phosphorylated AKT2 peptides in the native portion of the sample divided by the total mass of AKT2 peptides in the sample as indicated by the dephosphorylated portion of the sample.

Phosphorylation status can vary with the number of phosphorylation sites within a single protein or peptide that are phosphorylated, or with the number of proteins or peptides within a sample population that contain phosphorylation.

The detection or measurement of an elevated level of phosphorylation of AKT1 or AKT2, or both can be used as an indicator for using a PI3K/AKT/mTOR pathway inhibitor as a cancer therapy (e.g., patients with an elevated level of phosphorylation of AKT1 or AKT2, or both, can be selected for therapy with PI3K/AKT/mTOR pathway inhibitor). In an example, AKT1 phosphorylation status can be elevated independent of AKT 2 phosphorylation status, or vice versa. In another example, both AKT1 and AKT2 phosphorylation status can be elevated. In some examples, an elevated level of phosphorylation of AKT1 or AKT2, or both, is an amount of phosphorylation that is at least 50%, at least 75%, at least 100%, at least 200%, at least 300% or at least 500% more than that observed in a healthy patient population for the same protein(s) (e.g., those without cancer). A subject with such elevated levels AKT1 or AKT2, or both, phosphorylation can be administered can be selected and administered one or more PI3K/AKT/mTOR pathway inhibitors.

In an example, an elevated level of phosphorylation is greater than the level of phosphorylation of AKT1 or AKT2, or both, observed in a healthy patient population (e.g., those without cancer). In some examples, a healthy patient population has a level of phosphorylation of AKT1 or AKT2, or both of about 1%, 2%, 3%, 4%, 5%, 6%, 7%, 8%, 9%, 10%, 11%, 12%, 13%, 14%, 15%, 16%, 17%, 18%, 19%, or 20% phosphorylation. In an example, the percent level of phosphorylation is specific to the particular peptides assayed. For example, the peptides of SEQ ID NO: 1 and SEQ ID NO: 2 contain phosphorylation sites.

### Cancer Therapy

In an example, a patient exhibiting phosphorylation levels of AKT1, AKT2, or both, such as an increase of at least 10%, at least 15%, at least 20%, at least 25%, at least 30%, at least 35%, at least 40%, at least 45%, at least 50%, at least 55%, at least 60%, at least 70%, at least 80%, at least 90%, at least 100%, at least 2×, at least 3×, at least 4× or at least 5× greater than what is observed (or expected) in a healthy population (e.g., corresponding non-cancer sample of the same tissue type as the cancer sample, e.g., a normal breast tissue sample for a breast cancer test sample) or more are candidates for therapy with a PI3K/AKT/mTOR pathway inhibitor. Such subjects can be selected and administered one or more PI3K/AKT/mTOR pathway inhibitors.

The PI3K/AKT/mTOR pathway is a commonly dysregulated signaling pathway linked to cancer development and progression. Cancer therapeutics targeting the PI3K/AKT/mTOR pathway can be large or small molecules, or antibody therapeutics (such as biologics). One or more PI3K/AKT/mTOR pathway inhibitors can be used alone or in combination, or in concert with other chemotherapy, radiation and/or surgical therapies.

Example PI3K inhibitors that can be administered to a subject with increased phosphorylation levels of AKT1, AKT2, or both, include Wortmannin (CAS#19545-26-7), demethoxyviridin (a derivative of Wortmannin), LY294002 (CAS #154447-36-6), Idelalisib (ZYDELIG®), Perifosine (CAS #157716-52-4), Buparlisib (also known as BKM120), Duvelisib (also known as IPI-145), Alpelisib (also known as BYL719), TGR 1202 (also known as RP5264; CAS#1532533-67-7), Copanlisib (CAS #1032568-63-0), PX-866 (CAS #502632-66-8), Dactolisib (CAS #915019-65-7), ME-401 (also known as PWT143), IPI-549 (CAS#1693758-51-8), SF1126 (CAS#936487-67-1), RP6530 (CAS#1639417-53-0), INK1117 (CAS#1268454-23-4), pictilisib (CAS#957054-30-7), XL147 (CAS#956958-53-5), XL765 (CAS#1349796-36-6), Palomid 529 (CAS#914913-88-5), GSK1059615 (CAS#958852-01-2), PWT33597 (also known as VDC-597), CAL263 (Callistoga Pharmaceuticals), RP6503 (Rhizen Pharmaceuticals S.A.), PI-103 (371935-74-9), GNE-477 (1032754-81-6), or AEZS-136 (Aeterna Zentaris Inc.).

Example mTOR inhibitors that can be administered to a subject with increased phosphorylation levels of AKT1, AKT2, or both, include rapamycin (SIROMLIMUS™; CAS #53123-88-9), temsirolimus (CAS #162635-04-3), Everolimus (CAS #159351-69-6), or Ridaforolimus (CAS #572924-54-0).

Example AKT inhibitors that can be administered to a subject with increased phosphorylation levels of AKT1, AKT2, or both, are described in Nitulescu et al. Int J Oncol. 2016 March; 48(3): 869-885, and include H-8, H-89, NL-71-101, GSK690693, CCT128930, AT7867, AT13148, afuresertib, DC120, MK-2206, Edelfosine, Miltefosine, perifosine, Erucylphosphocholine, Erufosine, SR13668, OSU-A9, PH-316, PHT-427, PIT-1, PIT-2, DM-PIT-1, Triciribine, ARQ 092, or API-1. AKT inhibitors can be inhibitors of AKT1, AKT2, or both.

Inhibitors of the PI3K/AKT/mTOR pathways can further be used in combination with other agents for cancer therapy. Exemplary agents that can be used include one or more anti-neoplastic agents, such as radiation therapy, chemotherapeutic, biologic (e.g., immunotherapy), and anti-angiogenic agents or therapies. Methods and therapeutic dosages of such agents are known to those skilled in the art, and can be determined by a skilled clinician. These therapeutic agents (which are administered in therapeutically effective amounts) and treatments can be used alone or in combination. In some examples, 1, 2, 3, 4 or 5 different anti-neoplastic agents are used as part of the therapy.

In one example the additional therapy includes administration of one or more chemotherapy immunosuppressants (such as Rituximab, steroids) or cytokines (such as GM-CSF). Chemotherapeutic agents are known (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Exemplary chemotherapeutic agents that can be used with aPI3K/AKT/mTOR pathway inhibitor therapy include but are not limited to, carboplatin, cisplatin, paclitaxel, docetaxel, doxorubicin, epirubicin, cabaziatxel, estramustine, vinblastine, topotecan, irinotecan, gemcitabine, iazofurine, etoposide, vinorelbine, tamoxifen, valspodar, cyclophosphamide, methotrexate, fluorouracil, mitoxantrone, and Doxil® (liposome encapsulated doxiorubicine). In one example the additional therapy includes docetaxel and prednisone. In one example the additional therapy includes cabaziatxel.

In one example, the additional therapy includes administering one or more of a microtubule binding agent, DNA intercalator or cross-linker, DNA synthesis inhibitor, DNA and/or RNA transcription inhibitor, antibodies, enzymes, enzyme inhibitors, and gene regulators.

Microtubule binding agents interact with tubulin to stabilize or destabilize microtubule formation thereby inhibiting cell division. Examples of microtubule binding agents that can be used as part of the therapy include, without limitation, paclitaxel, docetaxel, vinblastine, vindesine, vinorelbine (navelbine), the epothilones, colchicine, dolastatin 10, nocodazole, and rhizoxin. Analogs and derivatives of such compounds also can be used. For example, suitable epothilones and epothilone analogs are described in International Publication No. WO 2004/018478. Taxoids, such as paclitaxel and docetaxel, as well as the analogs of paclitaxel taught by U.S. Pat. Nos. 6,610,860; 5,530,020; and 5,912,264 can be used.

The following classes of compounds can be used in combination with the PI3K/AKT/mTOR pathway inhibitor therapy: suitable DNA and/or RNA transcription regulators, including, without limitation, anthracycline family members (for example, daunorubicin, doxorubicin, epirubicin, idarubicin, mitoxantrone, and valrubicin) and actinomycin D, as well as derivatives and analogs thereof. DNA intercalators and cross-linking agents that can be administered to a subject include, without limitation, platinum compounds (for example, carboplatin, cisplatin, oxaliplatin, and BBR3464), mitomycins, such as mitomycin C, bleomycin, chlorambucil, cyclophosphamide, as well as busulfan, dacarbazine, estramustine, and temozolomide and derivatives and analogs thereof. DNA synthesis inhibitors suitable for use as therapeutic agents include, without limitation, methotrexate, 5-fluoro-5′-deoxyuridine, 5-fluorouracil and analogs thereof. Examples of suitable enzyme inhibitors include, without limitation, camptothecin, etoposide, exemestane, trichostatin and derivatives and analogs thereof. Suitable compounds that affect gene regulation include agents that result in increased or decreased expression of one or more genes, such as raloxifene, 5-azacytidine, 5-aza-2′-deoxycytidine, tamoxifen, 4-hydroxytamoxifen, mifepristone, and derivatives and analogs thereof. Kinase inhibitors include imatinib, gefitinib, and erolitinib that prevent phosphorylation and activation of growth factors.

In one example, the PI3K/AKT/mTOR pathway inhibitor therapy further includes folic acid (for example, methotrexate and pemetrexed), purine (for example, cladribine, clofarabine, and fludarabine), pyrimidine (for example, capecitabine), cytarabine, fluorouracil, gemcitabine, and derivatives and analogs thereof. In one example, the additional therapy includes a plant alkaloid, such as podophyllum (for example, etoposide) and derivatives and analogs thereof. In one example, the additional therapy includes an antimetabolite, such as cytotoxic/antitumor antibiotics, bleomycin, hydroxyurea, mitomycin, and derivatives and analogs thereof. In one example, the additional therapy includes a topoisomerase inhibitor, such as a topoisomerase I inhibitor (e.g., topotecan, irinotecan, indotecan, indimitecan, camptothecin and lamellarin D) or a topoisomerase II inhibitor (e.g., etoposide, doxorubicin, daunorubicin, mitoxantrone, amsacrine, ellipticines, aurintricarboxylic acid, ICRF-193, genistein, and HU-331), and derivatives and analogs thereof. In one example, the additional therapy includes a photosensitizer, such as aminolevulinic acid, methyl aminolevulinate, porfimer sodium, verteporfin, and derivatives and analogs thereof. In one example, the therapy includes a nitrogen mustard (for example, chlorambucil, estramustine, cyclophosphamide, ifosfamide, and melphalan) or nitrosourea (for example, carmustine, lomustine, and streptozocin), and derivatives and analogs thereof.

Other therapeutic agents, for example anti-tumor agents, that may or may not fall under one or more of the classifications above, also are suitable for use in combination with a PI3K/AKT/mTOR pathway inhibitor. By way of example, such agents include adriamycin, apigenin, rapamycin, zebularine, cimetidine, amsacrine, anagrelide, arsenic trioxide, axitinib, bexarotene, bevacizumab, bortezomib, celecoxib, estramustine, hydroxycarbamide, lapatinib, pazopanib, masoprocol, mitotane, tamoxifen, sorafenib, sunitinib, vandetanib, tretinoin, and derivatives and analogs thereof.

In one example, the additional therapy includes one or more biologics, such as a therapeutic antibody, such as monoclonal antibodies. Examples of such biologics that can be used include one or more of bevacizumab, cetuximab, panitumumab, pertuzumab, trastuzumab, bevacizumab (Avastin®), ramucirumab, and the like. In specific examples, the antibody or small molecules used as part of the therapy include one or more of the monoclonal antibodies cetuximab, panitumumab, pertuzumab, trastuzumab, bevacizumab (Avastin®), ramucirumab, or a small molecule inhibitor such as gefitinib, erlotinib, and lapatinib.

In some examples the additional therapy includes administration of one or more immunotherapies, which may include the biologics listed herein. In specific examples, the additional immunotherapy includes therapeutic cancer vaccines, such as those that target PSA (e.g., ADXS31-142), prostatic acid phosphatase (PAP) antigen, TARP, telomerase (e.g., GX301) or that deliver 5T4 (e.g., ChAdOx1 and MVA); antigens NY-ESO-1 and MUC1; antigens hTERT and survivin; prostate-specific antigen (PSA) and costimulatory molecules (e.g., LFA-3, ICAM-1, and B7.1) directly to cancer cells, such as rilimogene galvacirepvac. Other examples of therapeutic vaccines include DCVAC, sipuleucel-T, pTVG-HP DNA vaccine, pTVG-HP, JNJ-64041809, PF-06755992, PF-06755990, and pTVG-AR. In other examples, the immunotherapy includes oncolytic virus therapy, such as aglatimagene besadenovec, HSV-tk, and valacyclovir. In additional examples, the immunotherapy can include checkpoint inhibitors, such as those that target PD-1 (e.g., nivolumab, pembrolizumab, durvalumab, atezolizumab), CTLA-4 (e.g., tremelimumab and ipilimumab), B7-H3 (e.g., MGA271), and CD27 (e.g., CDX-1127). The protein MGD009 may also be used in another example. In specific examples, the immunotherapy can also include adoptive cell therapy, such as those that include T cells engineered to target NY-ESO-1 and those that include natural killer (NK) cells. In some examples, the immunotherapy can include adjuvant immunotherapies, such as sipuleucel-T, indoximod, and mobilan. In other specific examples, the immunotherapy includes one or more of tisotumab vedotin, sacituzumab govitecan, LY3022855, BI 836845, vandortuzumab vedotin, and BAY2010112, and MOR209/ES414. In additional examples, the immunotherapy can include cytokines, such as CYT107, AM0010, and IL-12.

In some examples, the subject receiving the PI3K/AKT/mTOR pathway inhibitor therapy is also administered interleukin-2 (IL-2), as part of the therapy, for example via intravenous administration. In particular examples, IL-2 is administered at a dose of at least 500,000 IU/kg as an intravenous bolus over a 15 minute period every eight hours beginning on the day after administration of the peptides and continuing for up to 5 days. Doses can be skipped depending on subject tolerance.

In some examples, the subject receiving the PI3K/AKT/mTOR pathway inhibitor therapy is also administered a fully human antibody to cytotoxic T-lymphocyte antigen-4 (anti-CTLA-4) as part of the therapy, for example via intravenous administration. In some example subjects receive at least 1 mg/kg anti-CTLA-4 (such as 3 mg/kg every 3 weeks or 3 mg/kg as the initial dose with subsequent doses reduced to 1 mg/kg every 3 weeks).

In one specific example for a subject with cancer (such as breast or colorectal cancer) the PI3K/AKT/mTOR pathway inhibitor therapy can further include administration of one or more of abiraterone acetate, bicalutamide, cabazitaxel, casodex (bicalutamide), degarelix, docetaxel, enzalutamide, flutamide, goserelin acetate, jevtana (cabazitaxel), leuprolide acetate, lupron (leuprolide acetate), lupron depot (leuprolide acetate), lupron depot-3 month (leuprolide acetate), lupron depot-4 month (leuprolide acetate), lupron depot-ped (leuprolide acetate), mitoxantrone hydrochloride, nilandron (nilutamide), nilutamide, provenge (sipuleucel-t), radium 223 dichloride, sipuleucel-T, taxotere (docetaxel), viadur (leuprolide acetate), xofigo (radium 223 dichloride), xtandi (enzalutamide), zoladex (goserelin acetate), and zytiga (abiraterone acetate).

In another specific example for a subject with cancer (such as breast or colorectal cancer), the PI3K/AKT/mTOR pathway inhibitor therapy can further include administration of one or more of chemotherapy drugs, such as cabazataxel (Jevtana®), docetaxel (Taxotere®), mitoxantrone (Teva®), or androgen deprivation therapy (ADT), such as with abiraterone Acetate (Zytiga®), bicalutamide (Casodex®), buserelin Acetate (Suprefact®), cyproterone Acetate (Androcur®), degarelix Acetate (Firmagon®), enzalutamide (Xtandi®), flutamide (Euflex®), goserelin Acetate (Zoladex®), histrelin Acetate (Vantas®), leuprolide Acetate (Lupron®, Eligard®), triptorelin Pamoate (Trelstar®). The therapy can also include drugs to treat bone metastases (bisphosphate therapy), such as alendronate (Fosamax®), denosumab (Xgeva®), pamidronate (Aredia®), zoledronic acid (Zometa®), or radiopharmaceuticals, such as radium 223 (Xofigo®), strontium-89 (Metastron®), and samarium-153 (Quadramet®).

The therapy can be administered in cycles (such as 1 to 6 cycles), with a period of treatment (usually 1 to 3 days) followed by a rest period. But some therapies can be administered every day.

### EXAMPLE 1

### Materials and Methods

This example provides the materials and methods used to obtain the results provided in the Examples below.

### Chemicals and Reagents

Chemicals and reagents were obtained from Fluka, Sigma Aldrich, and Thermo Fisher Scientific at the highest purities available, with the exception of TPCK-treated trypsin from Worthington Biochemical (89% purity). All organic solvents and H₂O were LC-MS grade (see Table 1). The purchased alpha-Cyano-4-hydroxycinnamic acid (HCCA) matrix was recrystallized as described below.

TABLE 1

| | Abbrevi- | | Catalog |
|---|---|---|---|
| Description | ation | Vendor | number |
| Acetonitrile (LC-MS grade) | ACN | Fluka | 34967 |
| Ammonium hydroxide | — | Fluka | 318612 |
| solution, 5.0M | | | |
| Water (LC-MS grade) | H₂O | Fluka | 39253 |
| Hydrochloric acid | HCl | Fluka | 84415 |
| fuming 37% | | | |
| 3-[(3-Cholamido- | CHAPS | Sigma | C9426 |
| propyl)dimethylammonio]- | | | |
| 1-propanesulfonate | | | |
| Alpha-cyano-4- | HCCA | Sigma | C2020 |
| hydroxycinnamic acid | | | |
| Ammonium bicarbonate | AmBic | Sigma | A6141 |
| Ammonium citrate dibasic | — | Sigma | A8170 |
| Dithiothreitol | DTT | Sigma | 43815 |
| Iodoacetamide | IAA | Sigma | I1149 |
| Phosphate buffered saline tablets | PBS | Sigma | P4417 |
| Sodium deoxycholate | DOC | Sigma | D6750 |
| Trifluoroacetic acid | TFA | Sigma | T6508 |
| TrisHCl, pH 8.1 | — | Sigma | T8568 |
| Bicinchoninic acid assay, | BCA | ThermoFisher | 23250 |
| reducing-agent compatible | assay | Scientific | |
| Bond-Breaker ™ TCEP | TCEP | ThermoFisher | 77720 |
| Solution, Neutral pH | | Scientific | |
| Halt phosphatase inhibitor | — | ThermoFisher | 78420 |
| cocktail | | Scientific | |
| Halt protease inhibitor | — | ThermoFisher | 78430 |
| cocktail | | Scientific | |
| Protein G Dynabeads ® | — | ThermoFisher | 10004D |
| | | Scientific | |
| Trypsin, TPCK-treated | — | Worthington | LS003744 |
| | | Biochemical | |
| | | Corporation | |

### Peptides, Recombinant Proteins and Antibodies

The unique C-terminal tryptic peptides of AKT1 (⁴⁶⁶RPHFPQFSYSASGTA⁴⁸⁰) and AKT2 (⁴⁶⁸ THFPQFSYSASIRE⁴⁸¹) were examined due to their involvement in full kinase activation (29, 30). Synthetic, light peptides (NAT) and stable isotope-labeled standard (SIS) peptides of these sequences were synthesized by using solid phase peptide synthesis, as previously described (31). Double-labeled versions (SIS-D) of these peptides were from SynPeptide (Beijing, China). Experiments showed that the AKT2 peptide is not cleaved at the R⁴⁸⁰ residue by trypsin. The AKT1 and AKT2 SIS and SIS-D peptides differ from the corresponding NAT peptides by 10 Da and 20 Da, respectively, due to the incorporation of stable isotope-coded arginine and phenylalanine residues (¹³C, ¹⁵N). After synthesis, the lyophilized peptides were resuspended in 30% acetonitrile (ACN)/0.1% formic acid (FA), and stored as stock solutions at −80° C. until used. The purities of the peptides were >86% as determined by capillary zone electrophoresis (CZE). Peptide concentrations were determined by amino acid analysis (AAA). Recombinant, full-length human AKT1 (ab116412) and AKT2 (ab79798) proteins were from Abcam (Cambridge, UK).

Polyclonal rabbit anti-AKT1 and anti-AKT2 were generated towards the sequences CRPHFPQFSYSASGTA and CTHFPQFSYSASIR (Intavis) as described (32). Briefly, a 4 mg/mL peptide solution was reduced with 1 equivalent of tris(2-carboxyethyl)phosphine (TCEP; Sigma-Aldrich) prior to conjugation with sulfo-m-maleimidobenzoyl-N-hydroxysuccinimide ester activated keyhole limpet hemocyanin (Pierce) (4 mg/mL) in PBS for 1 hour at room temperature. Rabbits were immunized with these conjugates and sacrificed after day 81 to obtain polyclonal serum. Antibodies were purified by peptide-affinity chromatography and desalted using an FPLC chromatography system (GE Healthcare) according to a standard antibody purification protocol.

### Cell Lines and Tumor Samples

BL21 E. coli cells. BL21 E. coli cells were grown overnight at 37° C. while shaking at 180 rpm, followed by pelleting and resuspension of the cells in PBS, pH 7.2.

SW480 cells. SW480 human colon adenocarcinoma whole cell lysate was purchased from Abcam (ab3957).

HCT116 colon cancer cells. 5×10⁶ cells of HCT116 colon carcinoma cell line were plated in a 75-cm² cell culture flask (Greiner) and cultured in Dulbecco's Modified Eagle Medium (PAA) supplemented with 2 mM glutamine (PAA) and 10% fetal calf serum (PAA) at 5% CO₂ atmosphere at 37° C. When the cells reached a density of 70%, they were rinsed twice with pre-chilled PBS. Cells were harvested using a rubber policeman and pelleted at 500×g and 4° C.

The cells were lysed and protein extraction was performed as described below.

MDA-MB-231 breast cancer cells. MDA-MB-231 breast cancer cells were obtained from ATCC and grown at the Jewish General Hospital (JGH, Montreal) in 10% RPMI media in the presence of 5% CO₂ at 37° C. Cells were starved overnight and cultured in the presence of 0.25% FBS. The next day, cells were incubated with 10 ng/mL human recombinant EGF (Invitrogen) in 0.25% FBS for 10 min at 37° C. Cells were harvested at 80% confluency for protein extraction. The cells were lysed and protein extraction was performed as described below.

Breast tumor samples. Primary breast tumor samples were obtained from the breast cancer tissue repository.

HCT116 mouse tumor xenograft. The HCT116 mouse tumor xenograft was generated by implanting 1×10⁶ HCT116 colon cancer cells into the flank of immuno-compromised BALB/C nude mice (Charles River). After tumor growth, tumor tissue was collected and snap frozen. Frozen primary breast tumor tissues and mouse xenograft tissues were transferred to a pre-chilled 10-cm plate and protein extraction was performed. The tissue was homogenized using the Precellys® Evolution at 4500-5500 rpm for 10-20 seconds.

### Total Protein Quantitation.

Protein concentrations of cell and tissue lysates were determined by bicinchoninic acid (BCA) assay, following the manufacturer's protocol (Thermo Fisher Scientific, 23250).

Automated iMALDI Procedure for the Quantitation of AKT1 (RPHFPQFSYSASGTA) and AKT2 (THFPQFSYSASIRE) Peptides.

A general overview of the iMALDI method is shown in FIG. 1. An Agilent Bravo liquid-handling robot equipped with 96-channel LT head, a gripper designed to grip 96-well plates, a 96-chimney tip wash station connected to an Agilent Peristaltic Pump Module, an Agilent Peltier Thermal Station, and an Agilent Orbital Shaking Station, was used to automate all liquid handling steps of the iMALDI sample preparation in a 96-well format. During the assay development, some experiments were performed in either fully manual, semi-automated, or fully automated modes. This will be indicated in the separate experiments described below. Further details on labware and accessories can be found in Table 2.

TABLE 2
Agilent Bravo labware and accessories

| | | Catalog |
|---|---|---|
| Description | Vendor | number |
| 1.1 mL deep well plate, | Axygen ® Scientific | P-DW-11-C-S |
| round bottom, | | |
| polypropylene | | |
| 12-well reagent reservoir, | Axygen ® Scientific | RES-MW12-LP |
| low profile | | |
| 96-well reagent reservoir, | Axygen ® Scientific | RES-SW96-LP |
| diamond bottom | | |
| 8-well reagent reservoir | Axygen ® Scientific | RES-SW8-HP |
| 500 μL deep well plate | Axygen ® Scientific | P-DW-500-C |
| twin.tec 96-well PCR plate | Eppendorf | 951020401 |
| 96-well PCR plate | Axygen ® Scientific | PCR-96-FS-C |
| 2 mL deep well plate, | Axygen ® Scientific | P-DW-20-C |
| round bottom, | | |
| polypropylene | | |
| 384-well microplate | Greiner Bio-One | 781201 |
| μFocus MALDI plate 2600 | Hudson Surface | PFB2226000 |
| μm | Technology | |
| Microflex holder for μFocus | Hudson Surface | HMM0101000 |
| MALDI plate 2600 μm | Technology | |
| Bravo holder for four μFocus | Custom made by | — |
| MALDI plates 2600 μm | Milroy Engineering | |
| | Ltd, Victoria BC | |
| 96LT 250 μL Bravo tips | Agilent Technologies | 19477-002 |
| DynaMag-96 Side Skirted | ThermoFisher | 12027 |
| Magnet | Scientific | |
| VP771RPM magnet | VP Scientific | VP771RPM |

### Digestion.

Cell and tumor lysates were thawed on ice and then manually diluted to 0.1 μg/μL with PBS/0.015% CHAPS (PBSC) in 2-mL MAXYMum recovery microcentrifuge tubes (Axygen Scientific). A 250-μL aliquot of diluted sample was manually transferred to an ice-cold 1.1-mL deep-well plate. The Agilent Bravo was then used to aliquot the samples into two wells of a new 1.1-mL deep-well plate (one for AKT1, and one for AKT2 analysis), resulting in 10 μg of total protein per well. Next, the Bravo was used to add 10 μL of denaturation buffer (10% sodium deoxycholate (NaDOC), w/v; 200 mM TrisHCl, pH 8.1; 0.74 mM TCEP) to each sample. Then, the plate was manually transferred to a 60° C. incubator for a 30-min incubation, followed by automated addition of 10 μL of 0.74 mM IAA solution. The plate was then kept in the dark for 30 min at room temperature, followed by the automated addition of 10 μL 0.74 mM DTT, and 10 μL of trypsin. Trypsin:total protein ratios varied throughout assay development and will be discussed in the results section. After digestion at 37° C. for 1 hour, the digested samples were placed on ice for 10 min.

### Antibody-Coupling to Magnetic Beads.

Magnetic Protein G Dynabeads (Thermo Fisher Scientific; 2.8 μm diameter; 30 μg/μL), and the anti-AKT1 peptide antibody or the anti-AKT2 peptide antibody, diluted with PBSC, were transferred to a 2-mL deep-well plate. The Agilent Bravo then washed the beads with 7×25% ACN/PBSC, and 3× with PBSC at a wash buffer/bead slurry volume ratio of 4:1. The procedure then used a DynaMag-96 Side-Skirted Magnet (Thermo Fisher Scientific) to pellet the beads to the side of the wells, with approximately 20 seconds in between washes. After the last wash, PBSC (using the same volume as the initial bead slurry volume) and 0.2 μg of either AKT1 or AKT2 antibody per μL of initial bead slurry volume were added to the beads and incubated for 1 hour at room temperature while shaking on the Bravo at 1300 rpm. This resulted in two sets of beads in two separate wells—anti-AKT1 peptide and anti-AKT2 peptide beads. After the incubation, the antibody beads were washed with 3× PBSC using the same volume as before the bead-antibody conjugation. The beads were then resuspended in PBSC, using a volume equal to 10× the volume of the initial bead slurry.

### Addition of SIS Peptide Solution to the Digests.

AKT1 and AKT2 SIS peptide standard solutions were prepared by diluting the stock solutions with PBSC, followed by automated addition of 10 μL of the appropriate SIS standard solution to the digestion samples, resulting in 2 fmol SIS per well.

### Affinity-Enrichment.

After digestion, the Bravo added 10 μL of resuspended antibody-beads to each SIS peptide-containing digest, resulting in 1 μL of initial bead slurry volume (30 μg beads+0.2 μg antibody) per well. The solution was incubated at room temperature while shaking at 1300 rpm for 1 hour on the Bravo's orbital shaker.

### Bead Washing and Spotting.

The bead washing procedure used three different wash buffers, which were aliquoted by the Bravo to the four 96-well quadrants of a 384-well microplate (Greiner) from reservoir plates (Axygen Scientific): 1) 15% ACN/PBSC, 2) 15% ACN/5 mM ammonium bicarbonate (AmBic), and 3) 5 mM AmBic. After the affinity-enrichment step, the digest plate was placed on the Bravo, to which two types of magnets had been added: a DynaMag-96 Side-Skirted Magnet, which pulled the beads to the well sides, and a VP771RPM magnet (VP Scientific), which was used to pull the beads to the bottom of a PCR plate. During this procedure, the sample solution was discarded and the beads were washed in the 1.1-mL deep-well plate with 100 μL of wash buffer, starting with one wash of 15% ACN/PBSC, and a second wash with 15% ACN/5 mM AmBic, using the orbital shaker for resuspending the beads. After a third wash, this time with 15% ACN/5 mM AmBic, the beads were re-suspended and transferred to a new 150 μL PCR plate (Eppendorf). This allowed re-suspension of the magnetic beads in a low volume (10 μL) of 5 mM AmBic prior to spotting the beads onto the MALDI plate. The 10-μL volume allowed for successful re-suspension of the magnetic beads, and this step could not be performed in a 1.1-mL deep-well plate due to the shape and size of the wells. The beads were then spotted onto four disposable 96-spot μFocus Microflex MALDI plates (Hudson Surface Technology), using a custom-made plate adapter (Milroy Engineering Ltd, Victoria BC) which allowed the four MALDI plates to be arranged in a microplate format. A small USB-powered fan was used to facilitate drying of the MALDI spots.

### Application of the MALDI Matrix and Washing of the MALDI Spots.

After the MALDI spots were dry, the Agilent Bravo was used to transfer 1.5 μL of the HCCA MALDI matrix solution (3 mg/mL HCCA, 1.8 mg/mL ammonium citrate, 70% ACN, and 0.1% TFA; prepared in a glass vial) from a 500 μL 96 deepwell plate (Axygen Scientific) to each sample spot. The acidic pH of the matrix solution elutes the peptides from the antibody beads and allows co-crystallization with the matrix molecules. After the matrix solution was dry, the Bravo washed each sample spot for a total of three washes with 6 μL of 7 mM ammonium citrate. After each wash, 0.2 μL of wash buffer remained on each spot which was allowed to dry prior to the next wash.

### MALDI-T OF Analysis.

MALDI-TOF analysis was performed on a Bruker Microflex LRF mass spectrometer. The μFocus MALDI plates were placed on a Microflex holder manufactured by Hudson Surface Technology (New Jersey, USA). One mass spectrum per spot was acquired by summing 1000 shots in either the positive-ion linear mode or the reflector mode, using a fixed laser intensity and a random walk of 15 shots at each raster spot. The ion suppression acquisition parameter was set to 1250 Da. The AutoXecute methods were generated in the Bruker FlexControl 3.3 software to automatically acquire data from the MALDI plates. Upon data acquisition, a FlexAnalysis 3.4 script was used for automatic smoothing, baseline subtraction, and internal calibration based on the SIS peaks. Mass spectra acquired in the reflector mode were internally calibrated using monoisotopic mass-to-charge (m/z) values, while mass spectra obtained in the linear mode were calibrated using the average m/z because of the increased peak width which encompassed the entire isotopic envelope. The expected monoisotopic and average m/z values are listed in Table 3.

TABLE 3
Expected m/z values for AKT1 and AKT2 target peptides
RPHFPQFSYSASGTA (SEQ ID NO: 1) and THFPQFSYSASIRE
(SEQ ID NO: 2).

| | | Monoisotopic | |
|---|---|---|---|
| | | m/z of | Average m/z |
| Target peptide | Sequence | [M + H]¹⁺ | [M + H]¹⁺ |
| AKT1 NAT | RPHFPQFSYSASGTA | 1652.78 | 1653.77 |
| AKT1 SIS | (SEQ ID NO: 1) | 1662.79 | 1663.78 |
| AKT1 SIS-D | | 1672.80 | 1673.79 |
| AKT2 NAT | THFPQFSYSASIRE | 1669.80 | 1670.80 |
| AKT2 SIS | (SEQ ID NO: 2) | 1679.81 | 1680.81 |
| AKT2 SIS-D | | 1689.81 | 1690.82 |

### Data Analysis.

Mass lists were generated in FlexAnalysis 3.4 using the centroid peak picking algorithm. Endogenous (END) peptide concentrations were calculated by multiplying the light/heavy peptide intensity ratios by the amount of SIS peptide spiked into each sample, using Microsoft Excel. Linear regression analyses were performed in R.

### Evaluation of Anti-AKT1 and Anti-AKT2 Peptide Antibodies.

In order to assess the functionality of the anti-AKT1 and anti-AKT2 peptide antibodies, 50 fmol of synthetic AKT1 and AKT2 NAT and SIS peptides were captured from PBSC. The recombinant AKT1 and AKT2 proteins were digested overnight at a total protein:trypsin ratio of 20:1 in PBSC and 100 μg E. coli lysate protein per replicate, followed by iMALDI analysis.

### Optimization Experiments

Washing of MALDI spots to improve sensitivity. One μL of a 1 fmol/μL AKT1 SIS peptide standard solution, prepared in 30% ACN/0.1% FA, was spotted onto a MALDI plate and dried, followed by the application of 1.5 μL of MALDI matrix. After the spots were dry, the spots were washed with either H₂O, 7 mM ammonium citrate, or 7 mM ammonium citrate/0.1% TFA by applying 1.5 μL of wash buffer onto the spots, waiting for 5 seconds, and removing the buffer. This was repeated for a total of five washes. Prior to the washes, and in between each wash, the MALDI plate was analyzed using the positive-ion reflector mode.

Digestion Time-course Study. To determine a digestion time that resulted in consistent digestion efficiency, a time-course digestion study was performed with manual sample preparation. Recombinant AKT1 and AKT2 were spiked into E. coli lysate (100 μg total protein per replicate), followed by tryptic digestion for 0, 0.5, 1, 2, 4, 6, 16, and 21 hours at 37° C. at a trypsin:total protein ratio of 1:5 (w/w) in 1.5 mL MAXYMum recovery microcentrifuge tubes (Axygen Scientific). Three replicates were performed per time point. After digestion, the tubes were placed on ice for 10 min to slow the digestion reaction, and then stored at −80° C. until the next day. After thawing on ice, 50 fmol AKT1 SIS peptide was added to each tube, and the solutions were transferred to a 300 μL PCR plate (Axygen Scientific), followed by affinity-enrichment as described above. Instead of shaking on the Bravo orbital shaker, however, the PCR plate was rotated on a Thermo Fisher Scientific Labquake rotator at 8 rpm. After that, six manual washes with 160 μL of 5 mM AmBic were performed. After manual application of the matrix, the dried spots were washed three times with 5 μL of 7 mM ammonium citrate prior to MALDI analysis in the positive-ion linear MALDI mode. The quantified peptide levels of each time point were compared to the 1-hour time using Microsoft Excel's student t-test to determine if there was significant difference.

Optimization of trypsin:total protein ratio. Due to the fact that different cell and tissue lysates have varying protein concentrations, and because each lysate has to be diluted to a common protein concentration prior to digestion, the protease inhibitor amounts in each sample vary. To assess the impact of protease inhibitor concentration on digestion efficiency, parental MDA-231 cell lysate, having a concentration (2.34 μg/μL) which was at the higher end of expected lysate protein concentrations, was diluted to 0.1 μg/μL. Varying amounts of 1× Halt protease inhibitors (0.04, 0.07, 0.10, or 0.40×) were spiked into the diluted cell lysates to simulate samples with varying initial protein concentrations. The samples were digested using different trypsin: total protein ratios (1:5, 1:2, 1:1, or 2:1, w/w). The subsequent affinity-enrichment was performed manually, but with the same bead, antibody and buffer compositions as stated above. After automated bead washing, spotting, and spot washing, the samples were analyzed in the positive-ion linear MALDI mode.

### Assay Validation

Linear range. The linear concentration range of the assay was determined by digesting 10 μg E. coli lysate (trypsin:total protein ratio of 2:1, w/w), followed by the addition of constant amounts of AKT1 and AKT2 SIS peptides, and varying amounts of AKT1 and AKT2 SIS-D peptides. Affinity-enrichment and bead washing was performed manually with the same antibody and bead amounts, buffers and volumes described above. MALDI analysis was performed in the positive-ion linear MALDI mode.

Accuracy testing. Analytical accuracy was assessed by spiking MDA-231 breast cancer cell lysate digest with varying amounts of AKT1 and AKT2 SIS-D (2, 4, and 8 fmol/well), and constant SIS (2 fmol/well). Calibration curves were generated by spiking varying amounts of AKT1 and AKT2 SIS-D and constant SIS into an E. coli digest, and used in calculating the SIS-D quantities in the breast cancer cell lysate. Digestions were performed at a trypsin:total protein ratio of 2:1 (w/w). The accuracy was calculated as a percentage of SIS-D quantified compared to the SIS-D amount spiked into the cell lysate. MALDI analysis was performed in the positive-ion linear MALDI mode.

Interference testing. Interference testing was performed in order to assess matrix effects by serially diluting parental and EGF-induced MDA-231 breast cancer cell lysates to 100, 50, 25, 12.5 and 6.25 ng/μL. Each dilution was treated as a separate sample. The trypsin:total protein ratio was 2:1 (w/w). MALDI analysis was performed in the positive-ion linear MALDI mode.

### HCCA Recrystallization Protocol.

Prepare a saturated HCCA solution by adding 100 mg HCCA to 10 mL of H₂O. Add 500 μL of 5M ammonium hydroxide until most of the acid dissolves. Slowly add 37% HCl to the solution until a large portion of the acid has precipitated (˜pH 2). Centrifuge at 4500×g for 5 min and remove the supernatant. Wash by adding 5 mL of 0.1M HCl and vortex. Centrifuge at 4500×g for 5 min and remove the supernatant. Repeat for a total of three washes. Resuspend in 500 μL of 0.1M HCl. Transfer to a 1.5 mL microfuge tube. Centrifuge for 5 min at 13,000×g. Dry matrix in a SpeedVac. Afterwards, store the dried matrix protected from light at 4° C.

### Protein Extraction.

The cell-containing media was transferred to a centrifuge tube followed by centrifugation at 4° C. for 5 min at 2,348×g. The resulting pellet was washed with 2 mL cold 1×PBS for a total of three washes. In between washes, the cells were centrifuged at 4° C. for 1 min at 13,523×g. After the last wash, 300 μL of ice-cold T-PER tissue protein extraction reagent (Thermo Fisher Scientific, Catalog number: 78510), containing 1× halt phosphatase inhibitor (Catalog number: 78428) and 1× halt protease inhibitor (Catalog number: 78430). Cell lysis was facilitated by sonication with 10 short pulses of 1 second/pulse. The lysate was placed on ice for 30 seconds, and the sonication step was repeated twice. The tube was then centrifuged for 5 min at 4° C. at 9,391×g. The supernatant was split into two new microfuge tubes, of which one contained 50 μL for BCA analysis. Both tubes were stored at −80° C. until analysis.

Protein Extraction from FFPE Tissues.

The following protocol was used for extraction of proteins from archival FFPE tissue slices (10 μm thickness) or FFPE tissue microarray (TMA) cores.

In a first step, three FFPE tissue slices or one to three TMA cores were transferred to a 1.5 mL microfuge tube. The samples were deparaffinised by adding 1 mL xylene substitute (Sigma), vortexing for 10 seconds, and incubating at RT for 10 minutes, followed by centrifugation at 16000×g for 2 minutes. The supernatant was discarded, and the step repeated twice.

Whereas FFPE tissue slices do not require additional homogenization, the FFPE TMA cores, due to their thickness, were placed in a mortar, frozen with liquid nitrogen, and then ground with a pestle. The resulting powder was resuspended in 1 mL ethanol and transferred to a new 1.5 mL microfuge tube.

The tissues were then rehydrated by sequential washes of 1 mL of 100%, 96% and 70% ethanol. The 96% and 70% ethanol washes were repeated twice. Each wash included a 10 second vortexing step, a 10-minute incubation at RT, and centrifugation at 16000×g for 2 minutes, followed by removal of the supernatant.

Next, 150 μL of the protein extraction buffer (0.05M TrisHCl, pH 8.1, 2% sodium deoxycholate, 10 mM TCEP, 1× Halt protease and phosphatase inhibitor cocktail) was added to the rehydrated tissue sample. The tube was incubated on ice for 5 min, followed by brief vortexing. On a Thermomixer, the tube was then incubated at 900 rpm at 99° C. for 20 min, then at 80° C. for 2 hours. After the incubation, the tube was placed on ice for 1 min. Next, the tube was centrifuged for 15 min at 14,000×g at 4° C. The supernatant was transferred to a new 1.5 mL microfuge tube. A 25 μL aliquot was stored in a separate tube at −80° C. until total protein content was determined by reducing agent-compatible bicinchoninic acid BCA assay (Thermo Fisher).

### EXAMPLE 2

### Assay Development

### Evaluation of Anti-AKT1 and Anti-AKT2 Peptide Antibodies.

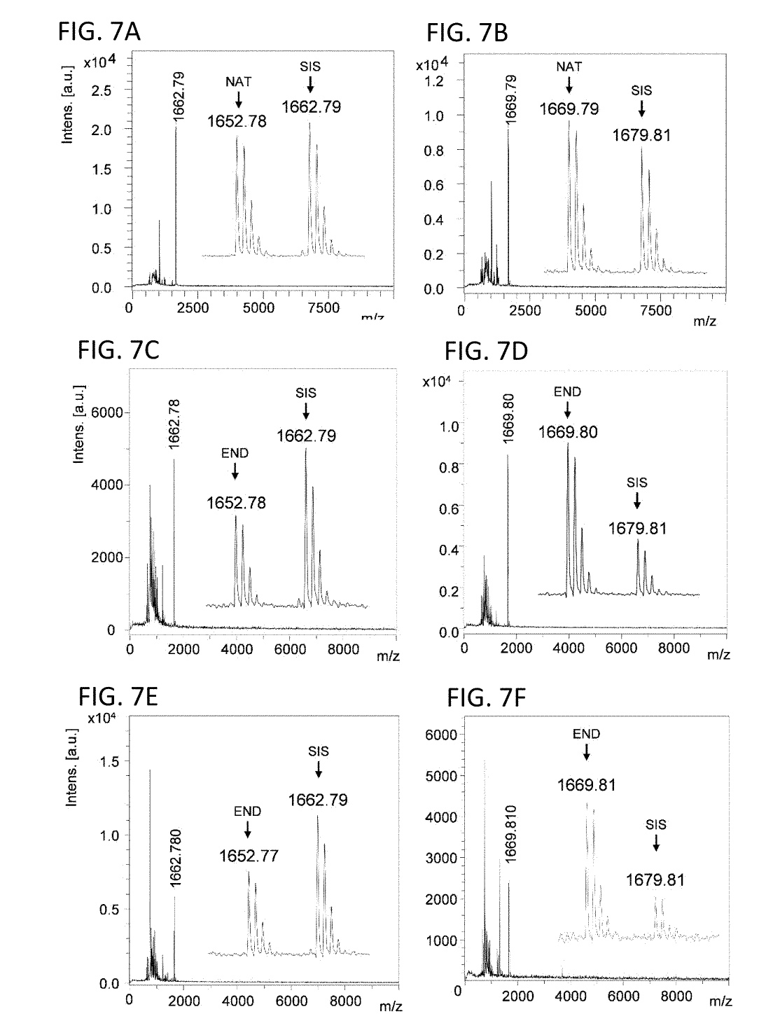

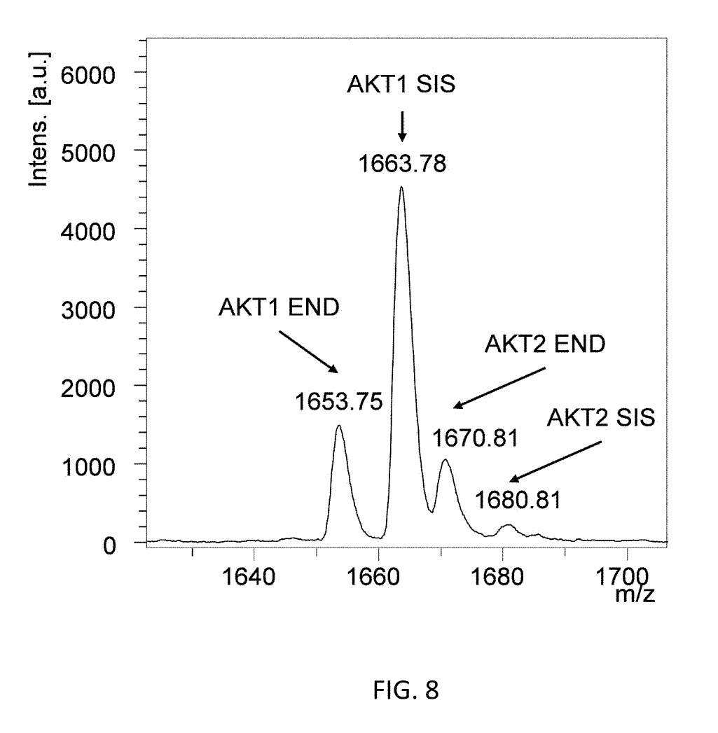

The functionality of the anti-AKT1 and anti-AKT2 peptide antibodies was evaluated by capturing AKT1 and AKT2 NAT and SIS peptides (FIGS. 7A and 7B), and peptides derived by tryptic digestion of recombinant AKT1 and AKT2 in PBSC (FIGS. 7C and 7D) or spiked into 100 μg E. coli lysate protein (FIGS. 7E and 7F). Each of the mass spectra show specific ion signals for NAT/END and SIS peptides, with no interfering signals, thereby demonstrating the suitability of the antibodies for capturing the target peptides from a simple buffer system as well as digested lysates, and the effectiveness of the sample preparation procedure in retaining the enriched target peptides while removing non-specific, potentially interfering compounds. Furthermore, while multiplexed analysis of several peptides is usually possible using the iMALDI technique—by adding magnetic beads carrying antibodies against different peptide targets to the same sample—the AKT1 SIS and AKT2 NAT ion signals show a slight overlap (FIG. 8). Thus, these peptides were quantified from two separate sample aliquots.

AKT1 Quantified from 100 μg of Cancer Cells and Flash-Frozen Tumor Lysates.

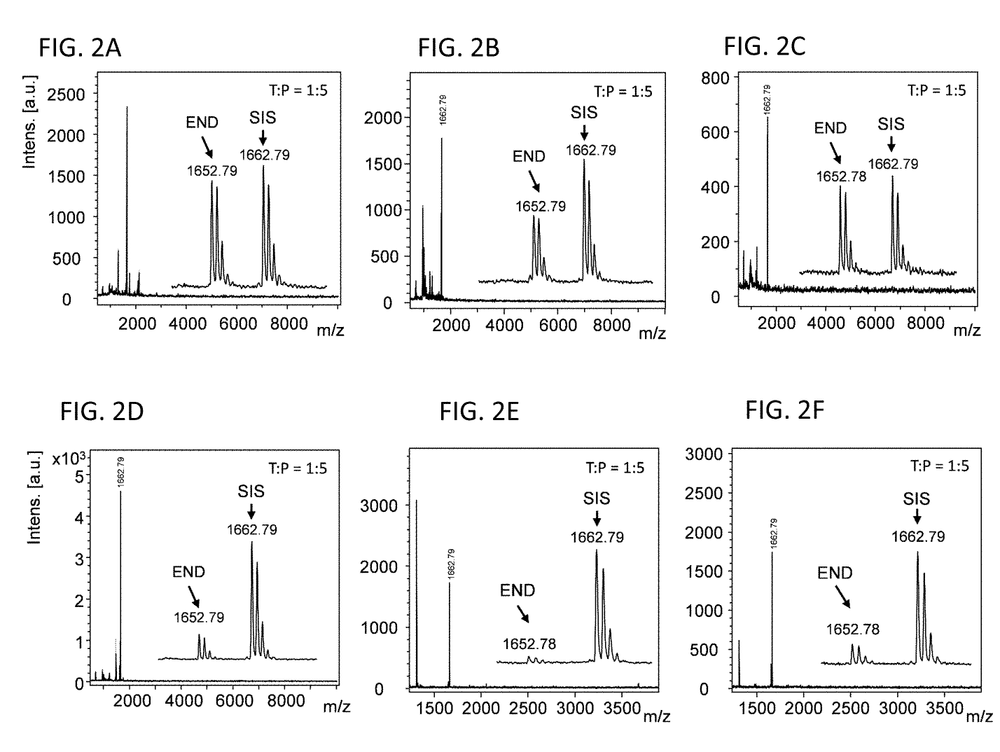

The suitability of the iMALDI protocol to detect the endogenous target peptides from complex matrices was assessed by analyzing AKT1 from 100 μg total protein of lysates of a breast cancer cell line (MDA-231, parental and EGF-induced), two colon cancer cell lines (SW480 and HCT116), and two flash-frozen breast tumors. FIG. 2A-FIG. 2F shows that the endogenous AKT1 target peptide were detected in all samples analyzed. The amounts quantified ranged from 29 amol/μg (1.6 pg/μg) for breast tumor 70-1 to 458 amol/μg (25.5 pg/μg) for the parental MDA-231 breast cancer cell line.

The total protein amount of 100 μg required per replicate is in the same range as other published MS-based quantitation methods that measure AKT1. However, the presented iMALDI AKT1 assay is the first MS-based method for absolute quantitation of AKT1 peptides from cancer tissues. In comparison, Atrih et al. analyzed AKT1 from 60 μg total protein of T-cells and a U-87 MG human primary glioblastoma cell line, using an approach that combines SDS-PAGE, in-gel digestion and MRM analysis (19). Just like the presented iMALDI assay, Atrih et al. targeted the ⁴⁶⁶RPHFPQFSYSASGTA⁴⁸⁰ (SEQ ID NO: 1) AKT1 peptide, encompassing the key phosphorylation sites S473, S477 and T479. However, gel-based assays are impractical and too tedious for a clinical setting. Another example of AKT1 quantitation by MS is an immuno-MRM or parallel reaction monitoring (PRM) approach by Patel et al, who have quantified several PI3K/AKT/mTOR pathway members, including AKT1, from 500 μg total protein of cancer cell lines (21). However, the feasibility both approaches have yet to be shown in tissue samples. In addition, the CPTAC assay portal lists two AKT1 (CPTAC-783 (33) and CPTAC-784 (34)) and two AKT2 (CPTAC-788 (35) and CPTAC-789 (36)) assays based on nanoLC separation followed by PRM analysis. The assays were validated in a pooled patient derived xenograft breast tumor digest matrix. However, the applicability of these assays for actual patient tumor samples has not been shown yet.

In summary, the experiments showed that the developed iMALDI protocol is able to quantify AKT1 from 100 μg total protein cancer cell lines and tumor samples. This, in theory, is sufficient to analyze needle biopsy samples, which, based on five in-house biopsy protein extractions, yielded 70-640 μg (median 139 μg). However, after removing approximately ⅔ of material for DNA/RNA extractions, only a minority of sample is left for protein extraction. Therefore, further assay optimization experiments were performed to improve the analytical sensitivity, thereby allowing a reduction in the sample amount required.

### EXAMPLE 3

### Assay Optimization

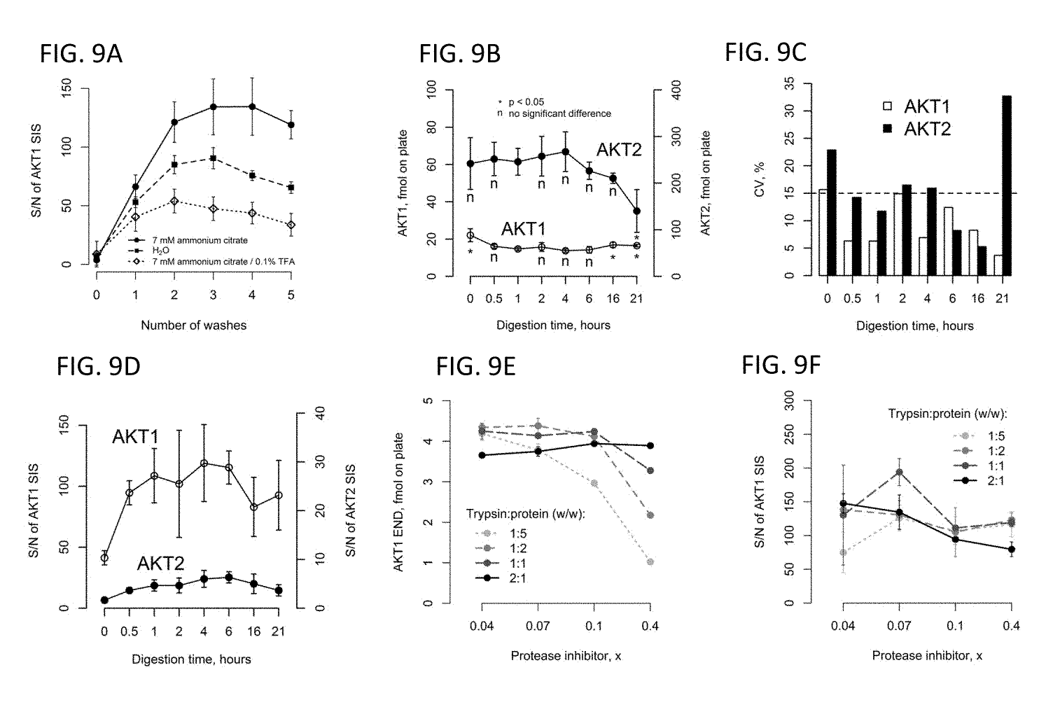

With the goal of improving sensitivity, various parameters were evaluated, including the effect of different sample dilution buffers, adjusting MALDI matrix composition, and adjusting wash buffers prior to spotting the magnetic beads onto the MALDI plate. However, these experiments only resulted in minor sensitivity improvements. In contrast, a significant improvement in sensitivity was achieved by washing the MALDI sample spots with a suitable buffer after matrix application and drying, prior to MALDI analysis. Of three wash buffers tested, washing the sample spots three or four times with 7 mM ammonium citrate solution led the largest signal-to-noise (S/N) increase (FIG. 9A). The S/N increased approximately 20-fold compared to no washing—from an average S/N of ˜6 to ˜130, whereas H₂O and 7 mM ammonium citrate/0.1% TFA led to increases of approximately 15-fold and 10-fold for three washes compared to no washes. Because of these results, all subsequent experiments were performed by washing the MALDI spots with 3×7 mM ammonium citrate prior to MALDI analysis. In addition, a protocol was created for the Agilent Bravo system that reproducibly washes up to 384 MALDI spots within ˜30 min.

Furthermore, a time-course digestion study of recombinant AKT1 and AKT2 spiked into 100 μg E. coli lysate (FIG. 9B-9D) demonstrated consistent digestion between 0.5 and 6 hours for AKT1, and 0 and 16 hours for AKT2. The digestion observed at the 0-hour time point can be explained by the rapid digestion occurring right after the addition of trypsin to the sample (prior to placing the sample on ice), and during the steps of the sample preparation at room temperature, such as the affinity-enrichment step. Based on these results, subsequent experiments were performed with a 1-hour digestion period.

Additionally, a trypsin:total protein ratio (w/w) of 2:1 was found to ensure consistent digestion efficiency that is independent of the protease inhibitor concentration in the sample (FIG. 9E) while maintaining comparable sensitivity compared to lower trypsin:total protein ratios (FIG. 9F).

### EXAMPLE 4

### Assay Validation

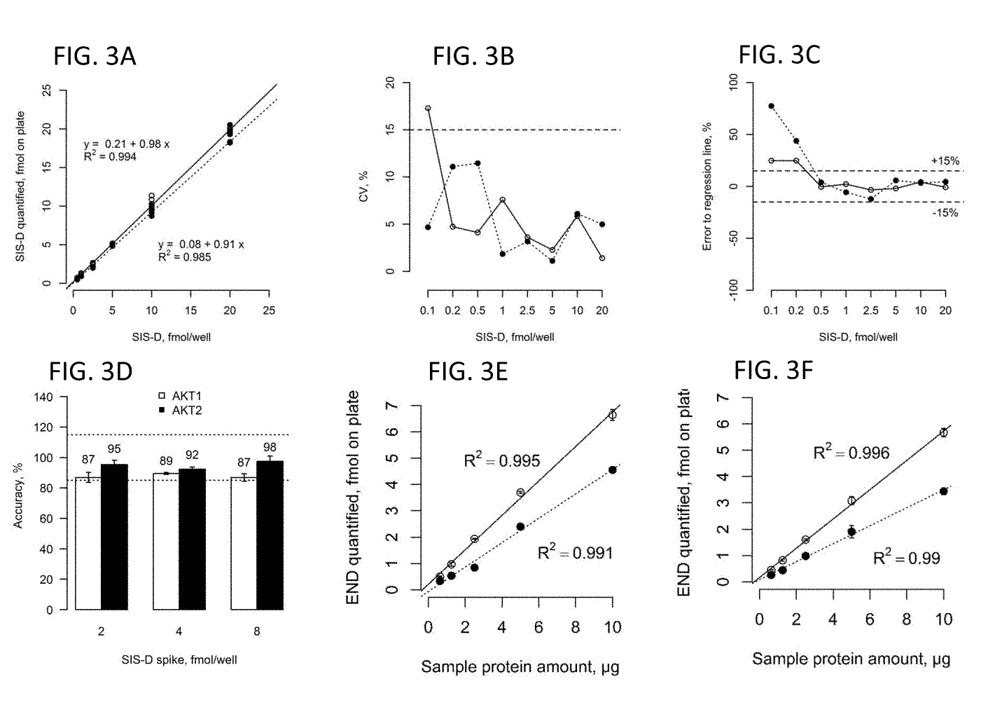

Linear range. The linear ranges of the AKT1 and AKT2 iMALDI assays were assessed by spiking different amounts of synthetic AKT1 and AKT2 SIS-D peptides and constant amounts of SIS peptides (2 fmol/well) into digests of 10 μg E. coli lysate. As can be seen from FIG. 3A, the 1/x²-weighted regression lines for the AKT1 and AKT2 peptides show excellent R² values of 0.994 and 0.985 and slopes of 0.98 and 0.91, respectively, demonstrating an excellent linear correlation of the spiked peptide concentration and the measured SIS-D/SIS intensity ratios. This shows that the intensity ratios can be used to measure the concentrations of the target peptides.

While the limit of detection (LOD) on the MALDI plate was 0.2 fmol of peptide for AKT1, the LOD on the MALDI plate for AKT2 was 0.5 fmol of peptide. For both AKT1 and AKT2, the % CV of all replicates between 0.2 and 20 fmol peptide on plate was below 15% (FIG. 3B), and the deviation of the mean amounts quantified at each point between 0.5 and 20 fmol peptide on plate were within ±15% (FIG. 3C), thereby meeting FDA criteria for bioanalytical method validation. (37) Taken together, the linear ranges for the AKT1 and AKT2 assays range from a lower limit of quantitation (LLOQ) of 0.5 fmol to an upper LOQ (ULOQ) of 20 fmol of peptide on the MALDI plate, corresponding to 2.8-111 pg/μg lysate protein for AKT1 and 2.6-102 pg/μg lysate protein for AKT2. All of the samples including fine needle biopsy analyzed to date fell within this range.

The absolute sensitivity of this iMALDI assays is comparable to other published approaches that combine immuno-enrichment with nanoLC-MS. For example, Whiteaker et al. (24) and Patel et al. (21) achieve LOQs of ˜2-8 fmol/mg equivalent to ˜1-4 fmol of AKT1, but each requires 500 μg total protein per analysis. The iMALDI assays presented herein achieve LOQs of 50 fmol/mg from only 10 μg total protein, thereby effectively achieving slightly higher absolute sensitivity of 0.5 fmol, while having the major benefit of enabling the analysis of small samples amounts, such as needle core biopsies.

Accuracy. The accuracy of the developed iMALDI assays was assessed by quantifying AKT1 and AKT2 SIS-D peptides spiked at three concentrations into MDA-231 cell lysate. A calibration curve generated in E. coli lysate as the surrogate matrix was used for quantification. The results in FIG. 3D show the accuracy values for AKT1 ranged from 87-89%, and for AKT2 from 92-98%, thereby falling within the acceptable ranges of 100±15% as specified by the FDA guidelines for bioanalytical method validation (37).

Interference testing. Levels of the endogenous AKT1 and AKT2 peptides quantified from serially diluted parental (FIG. 3E) and EGF-induced MDA-231 (FIG. 3F) cell lysates were plotted against sample protein amount and evaluated by linear regression. The endogenous peptide quantities and total sample protein amounts showed excellent correlation with R² values above 0.99, indicating the absence of significant interferences.

Analysis of AKT1/2 in biological samples from 10 μg total protein. After sensitivity optimization and switching from the positive-ion reflector MALDI mode to a positive-ion linear mode to improve further assay sensitivity and precision, the updated iMALDI procedure was tested on lysates of a breast cancer cell line (MDA-231, parental and EGF-induced), an HCT116 colon-cancer mouse xenograft tumor, and three flash-frozen breast tumor samples, using only 10 μg of lysate protein per quantitation replicate.

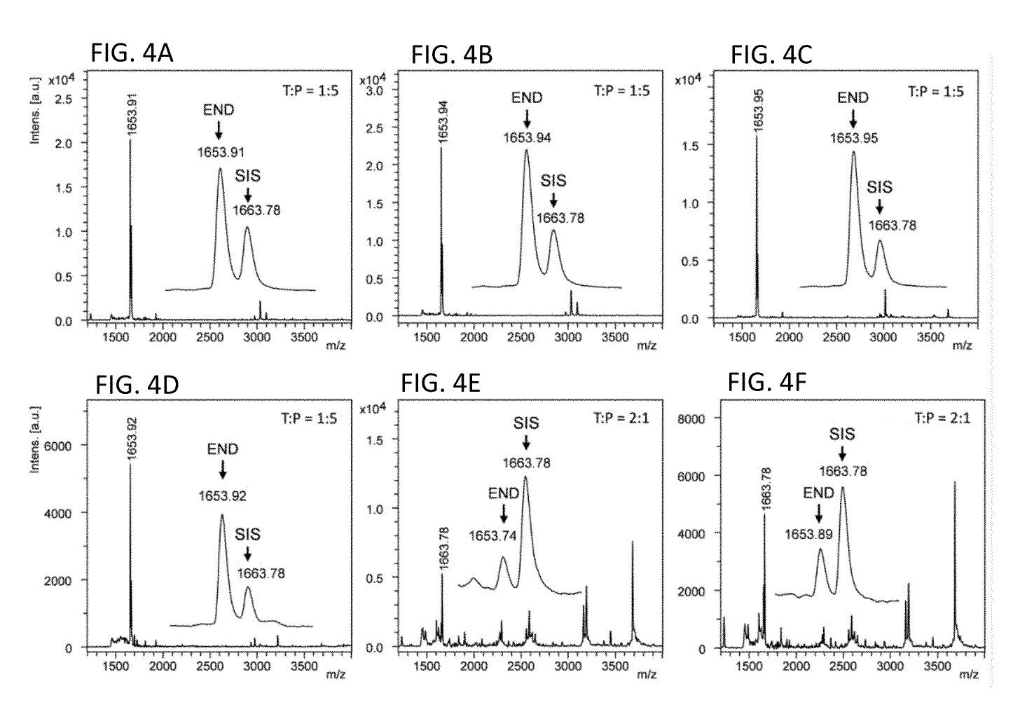

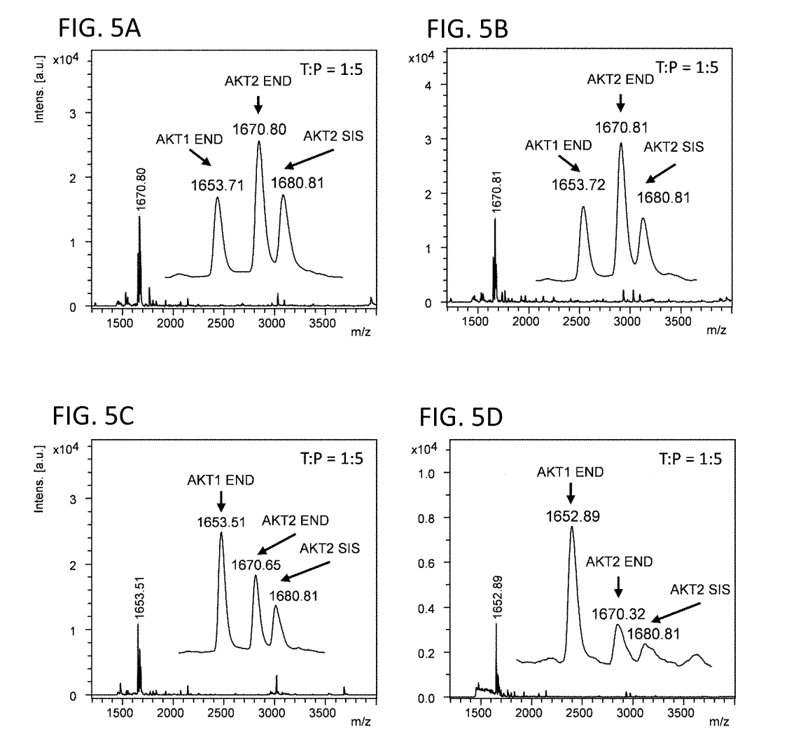

Endogenous AKT1 (FIG. 4A-4F) and AKT2 (FIG. 5A-5D) were detected in all samples analyzed. All values fell within the linear range of the developed assays. Especially noteworthy is the fact that in the breast tumor 70-1 sample shown in FIG. 2E, the AKT1 END peptide was barely detectable, whereas with the optimized procedure, the AKT1 END peptide could be clearly observed (FIG. 4E), even though the sample amount had been reduced from 100 μg to 10 μg total protein per replicate. The increased background peak abundances of the breast tumor samples 70-1 and 70-2 (FIGS. 4E and 4F), as compared to the other mass spectra in FIG. 4A-4D can explained by the elevated trypsin:total protein ratio of 2:1 which led to increased tryptic autolysis peptides binding non-specifically to the magnetic beads. However, no interfering peaks were observed in the range of the target peptides. This observation further demonstrates the great advantage of using MS for distinguishing between target and non-target peptides, thereby greatly reducing—if not eliminating—the chance of quantifying falsely elevated peptide levels.

Another advantage of combining affinity-enrichment with MS is the ability of enrich for multiple targets with a single, cross-reactive antibody. As can be seen in FIG. 5A-5D, all mass spectra not only show the AKT2 END and SIS peptides, but also the AKT1 END peptide. This is achieved by the cross-reactivity of the anti-AKT2 antibody that enriches both AKT1 and AKT2 target peptides. This phenomenon could be exploited by specifically developing and screening for cross-reactive antibodies for different isoforms of future target proteins.

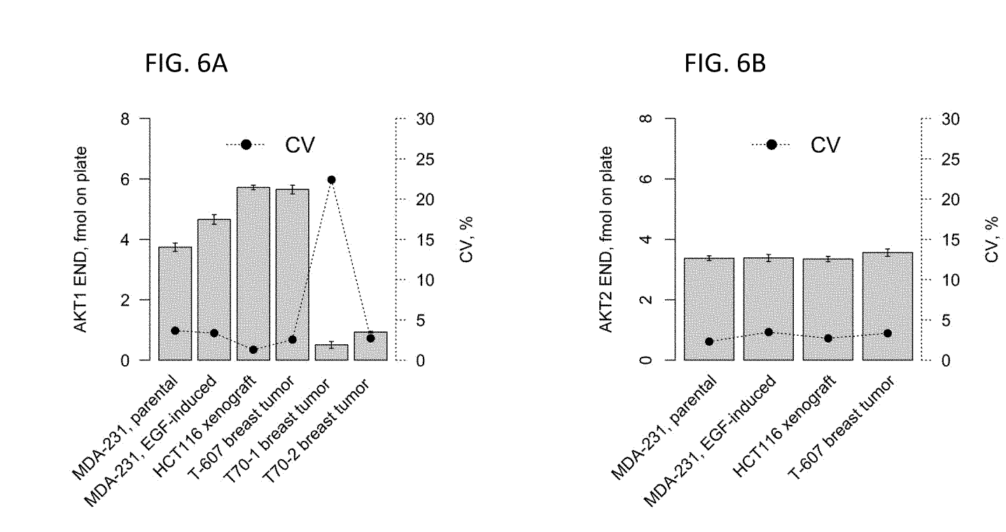

FIGS. 6A and 6B show the quantified AKT1 and AKT2 levels for the samples analyzed at 10 μg lysate protein per replicate, and Table 4 lists the corresponding values. The two samples with the lowest endogenous AKT1 levels are the breast tumor samples 70-1, and 70-2, with 51 and 93 amol AKT1/μg lysate, respectively. In contrast, the T-607 breast tumor and HCT116 colon cancer mouse xenograft tumor samples have approximately 10-fold higher AKT1 levels with 565 and 572 amol/μg, respectively. The AKT1 and AKT2 values quantified by iMALDI are very comparable to literature values for AKT1 and AKT2 presented by Patel et al. (21) who, as described above, used immuno-precipitation (IP) on the protein level from 500 μg of an A549 cell line lysate prior to digestion and quantitation on a nanoLC-MRM/PRM platform. Endogenous AKT1 and AKT2 levels were found to be in the range of ˜30-50 amol/μg lysate. Considering that the protein yield after enrichment is highly antibody-dependent and was determined by Patel et al. to be approximately 5% (38) the iMALDI vs. nanoLC-MRM/MS values are in good agreement, demonstrating the comparability of the two approaches.

TABLE 4
Quantified endogenous AKT1 and AKT2 peptide levels from
10 μg lysate protein of breast cancer cell lines, breast
tumors and an HCT116 colon cancer mouse xenograft tumor.

| | | Peptide amounts quantified per μg lysate | | | |
|---|---|---|---|---|---|
| | iMALDI | AKT1 | | AKT2 | |
| Sample | replicates | amol | pg | amol | pg |
| MDA-231, | 6 | 374 ± 14 | 21 ± 0.8 | 338 ± 7.8 | 19 ± 0.4 |
| parental | | | | | |
| MDA-231, | 6 | 466 ± 16 | 26 ± 0.9 | 338 ± 12 | 19 ± 0.7 |
| EGF-induced | | | | | |
| HCT116 | 3 | 572 ± 7.5 | 32 ± 0.4 | 335 ± 9.1 | 19 ± 0.5 |
| colon cancer | | | | | |
| mouse | | | | | |
| xenograft | | | | | |
| T-607 breast | 3 | 565 ± 14 | 31 ± 0.8 | 356 ± 12 | 20 ± 0.7 |
| tumor* | | | | | |
| Breast tumor | 4 | 51 ± 11 | 2.8 ± 0.6 | NP | NP |
| 70-1 | | | | | |
| Breast tumor | 4 | 93 ± 2.5 | 5.2 ± 0.1 | NP | NP |
| 70-2 | | | | | |

Furthermore, commercially available AKT1 and AKT2 ELISA assays that list quantified AKT1 and AKT2 concentrations in their product descriptions show comparable endogenous AKT1 levels from lysates of MCF7, HEK293 and HELA cells (1525, 388 and 730 amol AKT1/μg lysate protein) (Human AKT1 ELISA Kit (ab214023, ABCAM, Inc., Cambridge, UK)), and AKT2 levels from MCF7 lysate (875 amol AKT2/μg lysate protein) (AKT2 ELISA Kit (ab208986), ABCAM, Inc., Cambridge, UK)). The CVs for all samples analyzed, except one, were <5%, which is well within the precision criteria of 15% suggested by the FDA for bioanalytical method validation.(37)

### EXAMPLE 5

### Phosphorylation Detection Assay

General Workflow of the iMALDI-PPQ Method

The immuno-matrix assisted laser desorption/ionization phosphatase-based phosphopeptide quantitation (iMALDI-PPQ) approach allows quantitation of the expression levels and the phosphorylation stoichiometry of target proteins from cell line and tissue lysates. The general workflow is shown in FIG. 10. First, cell lysate is diluted with 20 mM TrisHCl/0.015% CHAPS to a final concentration of 0.1 mg/mL total protein. The diluted sample is then digested by addition of trypsin and incubation at 37° C. for 1 hour, followed by addition of the trypsin inhibitor N-Tosyl-L-lysine chloromethyl ketone hydrochloride (TLCK). Next, stable isotope-labelled peptide standards (SIS) analogous to the target peptide sequence, are added to the digested sample. The sample is then split into two aliquots, of which one is incubated at 37° C. for 2 hours with alkaline phosphatase. After the incubation, anti-peptide antibodies bound to magnetic beads specific for the non-phosphorylated target peptides are incubated for 1 hour at room temperature (RT). Next, the beads are washed to remove any residual non-specific compounds, and the beads-antibody-peptide complexes are directly spotted onto a MALDI plate. After the spots are dry, acidic alpha-Cyano-4-hydroxycinnamic (HCCA) MALDI matrix elutes the peptides from the beads and the peptides co-crystallize with the matrix molecules. MALDI analysis generates two spectra per sample: one for the sample aliquot that was treated with phosphatase, and one spectrum for the sample aliquot that was not treated with phosphatase. The endogenous levels of non-phosphorylated peptide in each aliquot is calculated by calculating the endogenous peptide-to-SIS (END/SIS) ratio. The END/SIS ratios of the unphosphorylated peptides of from both aliquots are then used to calculate the expression levels and phosphorylation stoichiometry of the target phosphopeptides in the sample.

### Optimization of Dephosphorylation Step

Two parameters were assessed to ensure complete dephosphorylation: phosphatase concentration, and dephosphorylation duration.

First, varying alkaline phosphatase (Roche, EIA grade) concentrations were added to three different backgrounds spiked with a synthetic pS473-AKT1 peptide: PBS+CHAPS buffer, an E. coli lysate digest, and an MDA-MB-231 breast cancer lysate digest. The dephosphorylation reaction was performed at 37° C. for 2 hours. Afterwards, the non-phosphorylated SIS standard was added, followed by the iMALDI procedure using the antibodies targeting the non-phosphorylated AKT1 peptide.

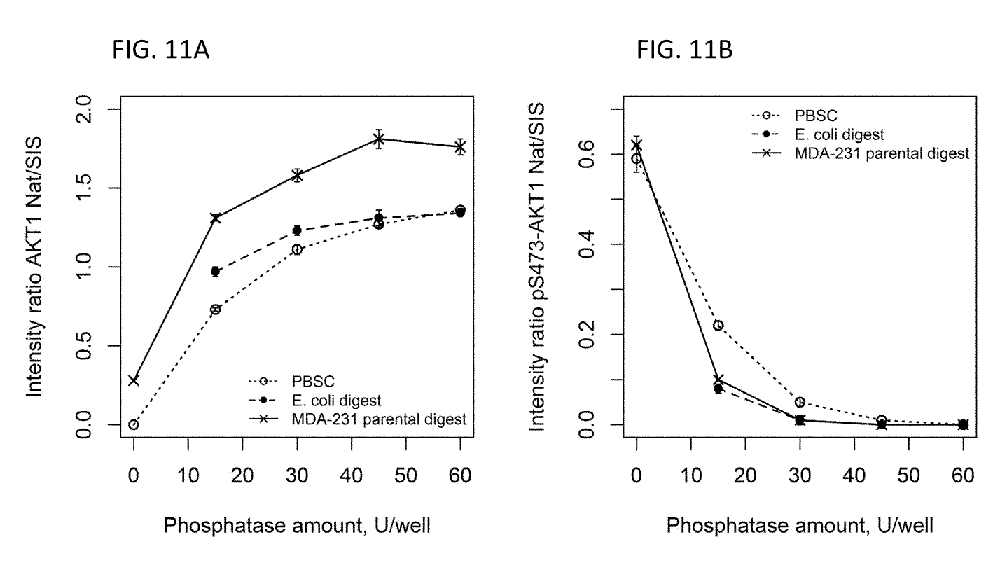

A synthetic pS473-AKT1 peptide was spiked into three different backgrounds and incubated with varying amounts of alkaline phosphatase (0 to 60 U/well). A phosphatase concentration of 60 U/well achieved complete dephosphorylation (FIGS. 11A and 11B). FIG. 11A shows an increase in non-phosphorylated AKT1 peptide generated by dephosphorylation of the synthetic phosphorylated peptide. The same trend, however by quantifying the light/heavy ratios of the light phosphorylated pS473-AKT1 peptide to heavy non-phosphorylated peptide can be seen in FIG. 11B. This is possible due to the cross-reactivity of the anti-AKT1 peptide antibody for both the phosphorylated and non-phosphorylated versions of the AKT1 peptide.

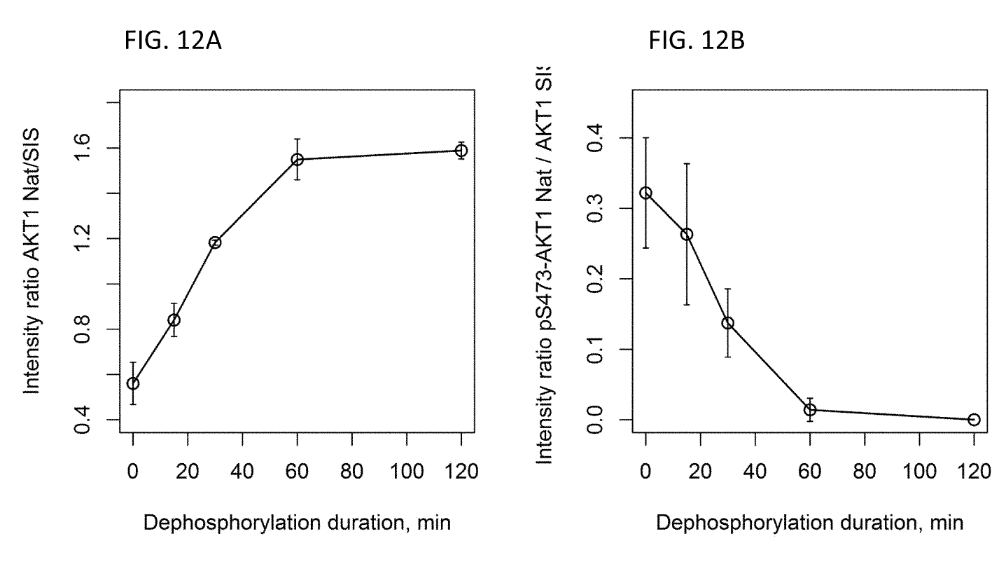

In a next step, the dephosphorylation duration was assessed by dephosphorylating the synthetic pS473-AKT1 peptide spiked into an MDA-MB-231 breast cancer cell lysate digest, and dephosphorylation at 60 U/well for 0 to 120 minutes. FIGS. 12A and 12B shows that 120 minutes achieve complete dephosphorylation at the phosphatase concentration used. Therefore, 60 U/well phosphatase and 120 minutes of incubating at 37° C. were determined to be the optimal parameters.

### EXAMPLE 6

### Quantitation of AKT1 and AKT2 Expression Levels and Phosphorylation Stoichiometry

Quantitation of AKT1 and AKT2 Expression Levels and Phosphorylation Stoichiometry from Cell Lines, Fresh Frozen and FFPE TMA Core Tissue Samples

To test the dephosphorylation step of the iMALDI-PPQ approach, MDA-MB-231 cell lysate, parental and EGF-induced, as well as on three fresh frozen tumor samples were analyzed for AKT1 with and without phosphatase treatment.

Furthermore, using the iMALDI-PPQ approach, FFPE TMA cores of normal and adjacent tumor tissues of two breast cancer patients were analyzed as well. The AKT1 and AKT2 target peptides in the patient samples were quantified with calibration curves prepared by spiking light synthetic peptides into E. coli lysate digests, followed by the iMALDI workflow.

Quantitation of AKT1 and AKT2 Expression Levels and Phosphorylation Stoichiometry from Cell Lines and Tumor Samples

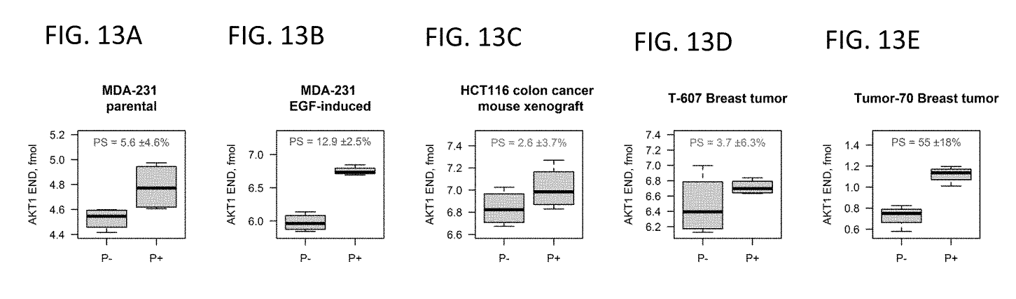

To assess the functionality of the dephosphorylation step of the iMALDI-PPQ procedure, the AKT1 target peptide was quantified from a breast cancer cell line and three fresh frozen tumor samples. AKT1 was quantified from all samples. Whereas the parental cell line resulted in phosphorylation stoichiometry of approximately 5% (FIG. 13A), the phosphorylation stoichiometry of the EGF-induced cell line is elevated at approximately 13% (FIG. 13B), and thereby meets the expectations that EGF induction leads to activation of AKT1. In comparison the HCT116 colon cancer mouse xenograft and T-607 breast cancer tissue (FIGS. 13C and 13D) showed minimal phosphorylation stoichiometry of <5%. In contrast, the tumor-70 breast tumor sample showed a phosphorylation stoichiometry of ˜55%. In conclusion, the iMALDI-PPQ procedure appears to be applicable to cell line and fresh frozen tissue analysis.

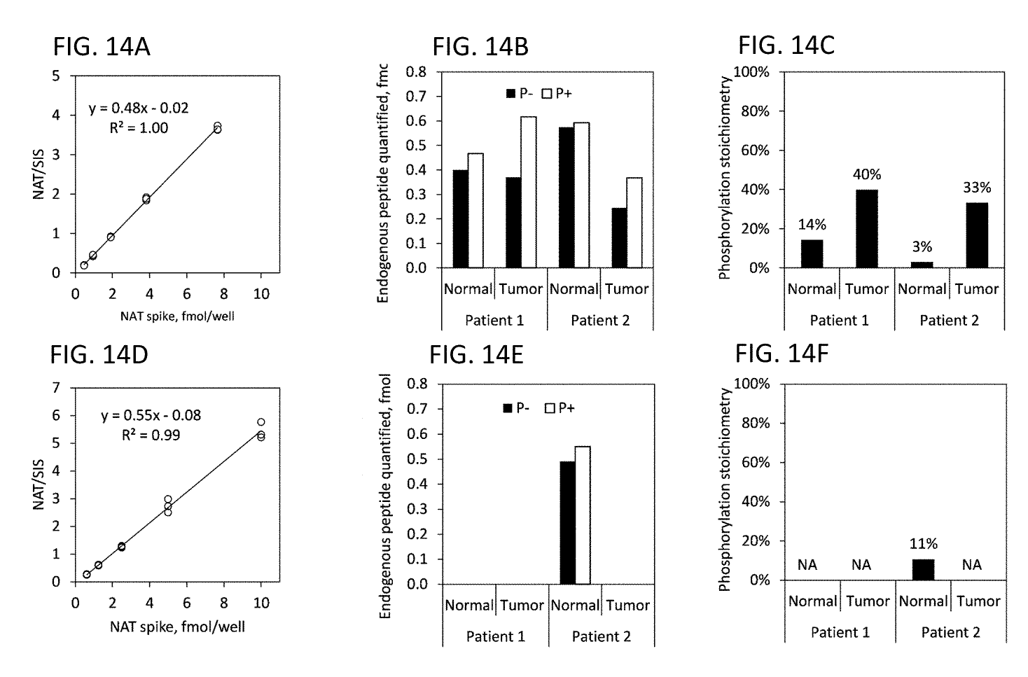

In order to further assess the functionality of the iMALDI-PPQ approach for FFPE tissue samples, which would allow retrospective studies of archival tissues existent in large numbers worldwide, normal and adjacent tumor tissues of two breast cancer patients were analyzed for AKT1 and AKT2 expression levels and phosphorylation stoichiometry. The calibration curves generated for quantifying the AKT1 and AKT2 target peptides in patient samples show excellent coefficients of determination of ≧0.99 (FIGS. 14A and 14C). The endogenous AKT1 levels for the four samples ranged from ˜0.2 fmol for the patient 2 tumor sample to ˜0.6 fmol for the normal patient 2 tissue (FIG. 14B). As shown in FIG. 14C, the patients' tumor samples show significantly elevated phosphorylation stoichiometries compared their corresponding normal tissues (40% vs. 14% for patient 1, and 33% vs. 3% for patient 2). In comparison, AKT2 values for only the normal tissue of patient 2 were obtained (FIGS. 14E and 14F), which could have been caused by protein degradation during the protein extraction procedure.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

### REFERENCES

1. de Gramont et al. Pragmatic issues in biomarker evaluation for targeted therapies in cancer. Nat Rev Clin Oncol 2015; 12(4):197-212.
2. Burrell et al. The causes and consequences of genetic heterogeneity in cancer evolution. Nature 2013; 501(7467):338-45.
3. Ruggles et al. An Analysis of the Sensitivity of Proteogenomic Mapping of Somatic Mutations and Novel Splicing Events in Cancer. Molecular & Cellular Proteomics 2016; 15(3):1060-71.
4. Kolch et al. The dynamic control of signal transduction networks in cancer cells. Nature Reviews Cancer 2015; 15(9):515-27.
5. Rivers et al. Linking cancer genome to proteome: NCI's investment into proteogenomics. Proteomics 2014; 14(23-24):2633-6.
6. Fruman D A, Rommel C. PI3K and cancer: lessons, challenges and opportunities. Nature reviews Drug discovery 2014; 13(2):140-56.
7. Polivka J, Janku F. Molecular targets for cancer therapy in the PI3K/AKT/mTOR pathway. Pharmacology & Therapeutics 2014; 142(2):164-75.
8. Ciriello et al. Emerging landscape of oncogenic signatures across human cancers. Nat Genet 2013; 45(10):1127-33.
9. Soung et al. Mutational Analysis of AKT1, AKT2 and AKT3 Genes in Common Human Carcinomas. Oncology 2006; 70(4):285-9.
10. Roy et al. AKT proto-oncogene overexpression is an early event during sporadic colon carcinogenesis. Carcinogenesis 2002; 23(1):201-5.
11. Cheng et al. Amplification of AKT2 in human pancreatic cancer cells and inhibition of ATK2 expression and tumorigenicity by antisense RNA. Proceedings of the National Academy of Sciences of the United States of America 1996; 93(8):3636-41.
12. Krześlak et al. Expression, Localization, and Phosphorylation of AKT1 in Benign and Malignant Thyroid Lesions. Endocrine Pathology 2011; 22(4):206-11.
13. Zhang et al. AKT2 expression is associated with glioma malignant progression and required for cell survival and invasion. Oncology Reports 2010; 24(1):65-72.
14. Rychahou et al. AKT2 overexpression plays a critical role in the establishment of colorectal cancer metastasis. PNAS 2008; 105(51):20315-20.
15. Chin et al. Targeting AKT3 signaling in triple-negative breast cancer. Cancer Res 2014; 74(3):964-73.
16. Ruggeri et al. Amplification and overexpression of the AKT2 oncogene in a subset of human pancreatic ductal adenocarcinomas. Mol Carcinog 1998; 21(2):81-6.
17. Baba et al., Phosphorylated AKT expression is associated with PIK3CA mutation, low stage, and favorable outcome in 717 colorectal cancers. Cancer 2011; 117(7):1399-408.
18. Dobashi et al., Diverse involvement of isoforms and gene aberrations of AKT in human lung carcinomas. Cancer Sci 2015; 106(6):772-81.
19. Atrih et al., Stoichiometric Quantification of AKT Phosphorylation Using LC-MS/MS. J Proteome Res 2009; 9(2):743-51.
20. Stokes et al. PTMScan direct: identification and quantification of peptides from critical signaling proteins by immunoaffinity enrichment coupled with LC-MS/MS. Mol Cell Prot 2012; 11(5):187-201.
21. Patel et al., Quantitative analysis of AKT/mTOR pathway using immunoprecipitation and targeted mass spectrometry. Cancer Research 2015; 75.
22. Parker C E, Borchers C H. Mass spectrometry based biomarker discovery, verification, and validation—Quality assurance and control of protein biomarker assays. Mol Oncol 2014; 8(4):840-58.
23. Addona et al. Multi-site assessment of the precision and reproducibility of multiple reaction monitoring-based measurements of proteins in plasma. Nat Biotechnol 2009; 27.
24. Whiteaker et al. Peptide Immunoaffinity Enrichment and Targeted Mass Spectrometry Enables Multiplex, Quantitative Pharmacodynamic Studies of Phospho-Signaling. Mol Cell Prot 2015; 14(8):2261-73.
25. Jiang et al.,Development of an immuno tandem mass spectrometry (iMALDI) assay for EGFR diagnosis. PROTEOMICS—Clinical Applications 2007; 1(12):1651-9.
26. Reid et al., Towards the development of an immuno MALDI (iMALDI) mass spectrometry assay for the diagnosis of hypertension. Journal of the American Society for Mass Spectrometry 2010; 21(10):1680-6.
27. Camenzind et al., Development and evaluation of an immuno-MALDI (iMALDI) assay for angiotensin I and the diagnosis of secondary hypertension. Clinical proteomics 2013; 10(1):20.
28. Popp et al. An automated assay for the clinical measurement of plasma renin activity by immuno-MALDI (iMALDI). Biochimica et biophysica acta 2015; 1854(6):547-58.
29. Liu et al., Cell-cycle-regulated activation of AKT kinase by phosphorylation at its carboxyl terminus. Nature (London) 2014; 508(7497):541-5.
30. Humphrey et al., High-throughput phosphoproteomics reveals in vivo insulin signaling dynamics. Nat Biotech 2015; advance online publication.
31. Kuzyk et al., Multiple Reaction Monitoring-based, Multiplexed, Absolute Quantitation of 45 Proteins in Human Plasma. Molecular & Cellular Proteomics 2009; 8(8):1860-77.
32. Hoeppe et al., Targeting peptide termini, a novel immunoaffinity approach to reduce complexity in mass spectrometric protein identification. Mol Cell Prot 2011; 10(2):M110.002857.
33. Office_of_Cancer_Clinical_Proteomics_Research_AKT1_Assay_Portal. AKT1, CPTAC-783. [cited 2017 Feb. 27, 2017]
34. Office_of_Cancer_Clinical_Proteomics_Research_AKT1_Assay_Portal. AKT1, CPTAC-784. [cited 2017 Feb. 24, 2017]
35. Office_of_Cancer_Clinical_Proteomics_Research_AKT2_Assay_Portal. AKT2, CPTAC-788. [cited 2017 Feb. 27, 2017]
36. Office_of_Cancer_Clinical_Proteomics_Research_AKT2_Assay_Portal. AKT2, CPTAC-789. [cited 2017 Feb. 27, 2017]
37. U.S. Food and Drug Administration. Guidance for Industry—Bioanalytical Method Validation. 2013.
38. Patel et al., Enrichment of EGFR/PI3K/AKT/PTEN Proteins for Research using Immunoprecipitation and with Mass Spectrometry-based Analysis. 62nd ASMS Conference on Mass Spectrometry and Allied Topics; 2014; Baltimore, Md.; 2014.

We claim:

1. A method of analyzing a sample obtained from a subject, comprising:
enzymatically digesting proteins in the sample, thereby producing a digested sample;
dephosphorylating a portion of the digested sample, thereby producing a dephosphorylated portion of the sample and a native portion of the sample;
contacting each portion of the sample with bead-antibody conjugates, wherein an antibody is specific for peptides of RAC-alpha serine/threonine-protein kinase (AKT1), RAC-beta serine/threonine-protein kinase (AKT2), or both AKT1 and AKT2, thereby producing bead-antibody conjugates bound to AKT1 and AKT2 peptides;
attaching the bead-antibody conjugates bound to AKT1 and AKT2 peptides onto a solid support;
washing the solid support; and
detecting the AKT1 and AKT2 peptides with mass spectrometry, thereby analyzing the sample

2. The method of claim 1, further comprising comparing the detected AKT1 and AKT2 peptides in the dephosphorylated portion of the sample to the AKT1 and AKT2 peptides in the native portion of the sample, thereby determining a phosphorylation status of AKT1 and AKT2.

3. The method of claim 1, wherein the sample is a cancer sample.

4. The method of claim 3, wherein the cancer sample is a colorectal cancer sample or breast cancer sample.

5. The method of claim 1, wherein enzymatically digesting the sample comprises contacting the sample with a proteolytic enzyme.

6. The method of claim 5, wherein the proteolytic enzyme is trypsin or ArgC.

7. The method of claim 6, wherein a ratio of the trypsin to total mass of protein in the sample in a range of 1.5:1 and 2.5:1 is used.

8. The method of claim 1, further comprising contacting the sample with stable-isotope-labeled standard (SIS) peptides following digesting the sample.

9. The method of claim 8, wherein the stable-isotope-labeled standard (SIS) peptides are isotope labeled RPHFPQFSYSASGTA (SEQ ID NO: 1) or THFPQFSYSASIRE (SEQ ID NO: 2).

10. The method of claim 1, wherein dephosphorylating a portion of the digested sample comprises contacting the digested sample with alkaline phosphatase.

11. The method of claim 10, wherein the digested sample is contacted with the alkaline phosphatase for about 2 hours.

12. The method of claim 10, wherein alkaline phosphatase is contacted with the digested sample at a concentration of 40-70 Units per 10 μg total protein.

13. The method of claim 1, wherein the antibody is specific for the peptides RPHFPQFSYSASGTA (SEQ ID NO: 1), or THFPQFSYSASIRE (SEQ ID NO: 2).

14. The method of claim 1, wherein washing the solid support comprises washing the dephosphorylated portion of the sample and the native portion of the sample with ammonium citrate or ammonium phosphate.

15. The method of claim 14, wherein the washing step is repeated three times.

16. The method of claim 14, wherein the ammonium citrate or the ammonium phosphate is incubated with the dephosphorylated portion of the sample and the native portion of the sample at a concentration of 1-20 millimolar.

17. The method of claim 1, further comprising administering a therapeutically effective amount of a cancer therapeutic to the subject, if a phosphorylation status is above 10%, 15%, 20%, 25%, 30%, 35%, 40%, 45%, 50%, 55%, 60%, or more % phosphorylation for AKT1 or AKT2.

18. The method of claim 17, wherein the cancer therapeutic is an inhibitor of PI3K, mTOR or AKT.

19. The method of any of claim 1, wherein the sample is fresh, frozen, or formalin-fixed paraffin-embedded (FFPE).

20. The method of claim 1, wherein each portion of the sample contains at least 10 μg total protein.
